(19) United States
(12) Patent Application Publication
(10) Pub. No.: US 2025/0161162 A1
(43) Pub. Date: May 22, 2025

(54) CONFIGURABLE ASSEMBLY FOR RETAINING AND DISPENSING LIQUIDS

(71) Applicant: Evolution Design & Development LLC, Perkasie, PA (US)

(72) Inventors: Anthony P. Pagliaro, JR., Perkasie, PA (US); Clemens E. Zoellner, Bay City, MI (US); Scott R. Johnson, Troy, NY (US)

(73) Assignee: Evolution Design & Development LLC, Perkasie, PA (US)

(21) Appl. No.: 19/028,348

(22) Filed: Jan. 17, 2025

Related U.S. Application Data
(63) Continuation-in-part of application No. 18/186,881, filed on Mar. 20, 2023.
(60) Provisional application No. 63/323,150, filed on Mar. 24, 2022.

(51) Int. Cl. A61J 1/12; A61M 39/10

(52) U.S. Cl. CPC A61J 1/12; A61M 39/1011

(57) ABSTRACT

A configurable port fitting for a container, such as a bioprocess bag, has a septa gasket groove and two opposing locking arms. The port fitting is mounted onto the container by (i) inserting one end of the port fitting into a port in the container such that a unitary septa gasket forms a seal between the port fitting and the container and (ii) rotating the port fitting to engage the locking arms with two opposing retention clips of the container. Some port fittings are fixed port fittings that provide a fixed opening into the container. Other port fittings are port sizers that receive a valve connector that enables flow control between closed and open positions. Port fittings of different sizes enable different flow rates. Some port fittings (and other connectors) have a threaded tube barb that receives a threaded tube retainer that secures tubing in place over the barb.

### CROSS-REFERENCE TO RELATED APPLICATIONS

This application is a continuation-in-part of U.S. patent application Ser. No. 18/186,881, filed on Mar. 20, 2023, which claims the benefit of the filing date of U.S. provisional application No. 63/323,150, filed on Mar. 24, 2022 as attorney docket no. 1405.001PROV, the teachings of all of which are incorporated herein by reference in their entirety.

### BACKGROUND

### Field of the Disclosure

The present disclosure relates to assemblies for retaining and dispensing liquids and, more specifically but not exclusively, to bioprocess bag assemblies, bottles, carboys, and cap assemblies, such as (without limitation) those used in medical, laboratory, bioprocessing, and cell and gene therapy applications.

### Description of the Related Art

This section introduces aspects that may help facilitate a better understanding of the disclosure. Accordingly, the statements of this section are to be read in this light and are not to be understood as admissions about what is prior art or what is not prior art.

Containers (e.g., flexible bags) for fluid containment have been used in numerous applications including bioprocess applications that require a high degree of assurance that (i) contamination will not enter the closed system and (ii) leakage will not occur.

### BRIEF DESCRIPTION OF THE DRAWINGS

Embodiments of the disclosure will become more fully apparent from the following detailed description, the appended claims, and the accompanying drawings in which like reference numerals identify similar or identical elements.

FIG. 1 is a plan view of a bag assembly 102 according to one embodiment of the present disclosure;

FIG. 2 is a plan view of the bag 104 of FIG. 1 without the bag hangar 106;

FIG. 3 is an enlarged, perspective, X-ray view of the bottom of the bag assembly 102 of FIG. 1;

FIG. 4A is a perspective, bottom-up view of the canoe assembly 402 of FIG. 1;

FIG. 4B is an end view of the canoe assembly 402 of FIG. 4A;

FIG. 4C is a perspective view of the canoe assembly 402 with the three valve connectors 110(1)-110(3) maintained at their “closed” configurations by three tethered safety clips 406;

FIG. 4D is a perspective view of the canoe assembly 402 with the three tethered safety clips 406 disengaged and with the three valve connectors 110(1)-110(3) at their “open” configurations;

FIG. 4E is an exploded, side view of the canoe assembly 402 of FIG. 4C;

FIG. 5 is a perspective view showing the bottom of the canoe 114;

FIG. 6A is a perspective view of the port sizer (AKA port fitting) 108(3);

FIG. 6B is a cross-sectional side view of the port sizer 108(3);

FIG. 6C is a perspective bottom view of the port sizer 108(3);

FIG. 7A is a perspective view of the large valve connector 110(3);

FIG. 7B is a cross-sectional side view of the large valve connector 110(3);

FIGS. 8A-8C show perspective views of small, medium, and large, unthreaded, fixed port fittings 802(1)-802(3), respectively, each of which can be used instead of a corresponding port sizer 108/valve connectors 110 pair;

FIGS. 8D-8F show perspective views of small, medium, and large, threaded, fixed port fittings 808 having threaded tube barbs 810 and stops 612;

FIG. 8G shows a side view of a fixed port fitting 814 having an integral, threaded “wye” tube connector with threaded tube barbs;

FIG. 8H shows a side view of a fixed port fitting 816 having an integral, threaded “tee” tube connector with threaded tube barbs;

FIG. 8I shows a perspective view of a fixed port fitting 802A;

FIG. 9 is a perspective view of a luer lock valve connector 902 having a luer lock 904 instead of a tube barb for mating with a tube (not shown);

FIG. 10 is a side view of block-out plug 1002;

FIGS. 11A-11C respectively show a “tee” valve connector 1102 with two threaded tube barbs 810, a “wye” valve connector 1104 with two threaded tube barbs 810, and a “wye” valve connector 1106 with two valve-connector housings 1108;

FIGS. 12A and 12B are plan views of female and male bag hangar components 1202 and 1204, respectively, which can be used to form the bag hanger 106 of FIG. 1;

FIG. 12C shows a perspective view of the female and male bag hangar components 1202 and 1204 positioned to be configured to the top portion of a bag chamber 112;

FIG. 12D shows a zoomed-in, perspective view of the male bag hangar 1204 configured with the bag chamber 112;

FIG. 12E shows a zoomed-in perspective view of the final bag hangar assembly;

FIG. 13 shows a side view of the bag assembly 102 of FIG. 1 with a bag probe 1302 attached to one of the valve connectors 110;

FIG. 14A is a perspective view of a canoe assembly 1400 having a tube retainer design in its “closed” configuration;

FIG. 14B is a cross-sectional perspective side view of the canoe assembly 1400 of FIG. 14A;

FIG. 14C is a cross-sectional perspective end view of the canoe assembly 1400 of FIG. 14A;

FIGS. 14D-14F are analogous to the views of FIGS. 14A-14C, respectively, but with the canoe assembly 1400 in its “open” configuration with the ends of the valve connectors 110 extending above the top surface of the canoe 114;

FIG. 15A is a cross-sectional end view of the canoe assembly 1400 of FIG. 14A in its “closed” configuration, and FIG. 15B is a cross-sectional end view of the canoe assembly 1400 of FIG. 14D in its “open” configuration;

FIG. 16A is a zoomed-in, cross-sectional side view of the canoe assembly 1400 of FIG. 14A with the port sizer locking clips 606 engaged with the valve connector “closed” locking tabs 706;

FIG. 16C is a zoomed-in, cross-sectional side view of the canoe assembly 1400 of FIG. 14D with the port sizer locking clips 606 engaged with the valve connector “open” locking tabs 708;

FIG. 16B is a zoomed-in, cross-sectional side view of the same canoe assembly 1400 transitioning from the “closed” configuration of FIG. 14A to the “open” configuration of FIG. 14D;

FIGS. 17A and 17B show perspective views of the canoe 114 and the port sizer 108(3), respectively;

FIGS. 17C and 17D show different, zoomed-in, cutaway, perspective views, and FIG. 17E shows a zoomed-in, perspective view of the canoe 114 and port sizer 108(3) of FIGS. 17A and 17B;

FIGS. 18A-18C show different stages of connecting different sized tubes 1802 to different sized, threaded, fixed port fittings 808 using different sized, threaded tube retainers 1404;

FIG. 19A is a side view of half of a threaded fixed port fitting 808, and FIG. 19B is a cross-sectional side view of the threaded fixed port fitting 808 of FIG. 19A;

FIG. 20A shows a perspective view of a port sizer 108, a valve connector 110, and a tethered safety clip 406 before they are assembled together;

FIG. 20B shows a perspective view of the elements of FIG. 20A after the tether ring 408 has been placed over the port sizer stop 612;

FIG. 20C shows a perspective view of the elements of FIG. 20B after the tether ring 408 has been rotated clockwise about 90 degrees with respect to the port sizer 108;

FIG. 20D shows a perspective view of the elements of FIG. 20C after the tether clip 410 has been rotated 180 degrees with respect to the tether ring 408, resulting in a bending of the tether 412;

FIG. 20E shows a perspective view of the elements of FIG. 20D and a dust cap 414 after (i) the valve connector 110 has been inserting into the port sizer 108 up to the “closed” configuration and (ii) the tether clip 410 has been pushed onto the valve connector 110 just below the port sizer stop 612 and just above the valve connector stop 710;

FIG. 20F shows a perspective view of the elements of FIG. 20E and a tube retainer 414 after (i) the dust cap 414 has been placed over the barbed end of the threaded tube barb 810 (not visible in FIG. 20F) and (ii) a tube retainer 1404 has been screwed onto the threaded tube barb 810;

FIG. 21 shows a perspective view of the bottom of a canoe assembly 1400 having three port sizer/valve connector sub-assemblies in three different configurations;

FIG. 22A is a perspective view of a tube retainer 1404, and FIG. 22B is a cross-sectional perspective view of the tube retainer 1404 of FIG. 22A;

FIG. 23 is a zoomed-in cross-sectional perspective view of a tube retainer X046 screwed onto a threaded tube barb 810 with an intervening dust cap 414;

FIG. 24 is a cross-sectional side view of a tube retainer 1404 fully screwed onto a threaded tube barb 810 with a tube 1802 secured and sealed in place;

FIG. 25 is a perspective view of a canoe assembly 2502 having a two-port canoe 2504;

FIG. 26A is an exploded perspective view of a single-fitting port 2602 configured to receive a single port fitting;

FIG. 26B is a top-down view of a two-fitting port 2616 configured to receive two port fittings, and FIG. 26C is a top-down view of a four-fitting port 2618 configured to receive four port fittings;

FIGS. 26D and 26E are side and cross-sectional (through the center line) views, respectively, of the two-fitting port 2616 of FIG. 26B with two fixed port fittings 2604 and a septa gasket 2614;

FIGS. 27A and 27B are exploded and assembled views, respectively, showing a side view of the fixed port fitting 2604 and a cross-sectional view of a septa gasket 2614;

FIG. 27C is a cross-sectional side view of the fixed port fitting 2604;

FIG. 28 is a partially exploded view of the fixed port fitting 2604 and the single-fitting port 2602 mated with a cap 2802 for a bottle or carboy;

FIG. 29A is a side view of a dip tube 2902 having a dip tube fitting 2904 with two O-ring grooves 2906, and FIG. 29B is a side view of a dip tube 2908 having a dip tube fitting 2910 with a single septa gasket groove 2912;

FIG. 30A is a perspective view of a filling needle 3002, FIG. 30B is a perspective view of the filling needle 3002 with a dust cap 414, and FIG. 30C is a perspective view of the filling needle 3002 with both the dust cap 414 and a threaded tube retainer 1404;

FIGS. 30D-30F are perspective views representing a tube 1802 being mounted onto the filling needle 3002 of FIGS. 30A-30C using the threaded tube retainer 1404;

FIG. 31 is a side view of a vented port fitting 3102;

FIGS. 32A, 32B, 32C, and 32D show top-down, end, X-ray side, and cross-sectional side views of a three-port canoe 3202 according to an alternative embodiment;

FIG. 32E is an exploded side view of the three-port canoe 3202 of FIGS. 32A-32D ready to receive three fixed port fittings 2604;

FIGS. 33A and 33B are side views of two different tube-to-tube connectors;

FIG. 33C is a side view of a two-piece tube-to-tube connector 3306 that provides flow-valve functionality;

FIGS. 33D-33F show side views of retainer-ready cross, tee, and wye tube-to-tube connectors, respectively;

FIGS. 33G and 33H show side views of retainer-ready tri-clamp tube barb connectors;

FIGS. 33I and 33J show side views of two different three-piece tube-to-tube TEE connectors that provide flow-valve functionality at their lower ports;

FIG. 33K is a side view of the three-port tube-to-tube connector analog 3312 of FIGS. 33I and 33J with a probe 3314 connected to the lower port to enable fluid path measurements;

FIG. 34 is a side view of a fixed port fitting 3402 having a threaded tube barb with three tube grippers 3404;

FIG. 35 is an exploded side view of a reconfigurable assembly 3502;

FIG. 36 is a perspective view of the bottom of a bag assembly 3602 according to an alternative embodiment;

FIG. 37 is an end view of the port fitting 3608(3) of FIG. 36 fully mounted into the third port 3612(3) of the canoe 3604;

FIGS. 38A and 38B are a perspective view and a plan view, respectively, of the three-port canoe 3604 of FIG. 36;

FIG. 39A is a plan view of a one-port 3D bag connector 3902, FIG. 39B is a plan view of a three-port 3D bag connector 3904, and FIG. 39C is a perspective view of a two-port bottle cap connector 3906;

FIG. 40A is a side view of the port fitting 3608(3) of FIG. 36 configured with a so-called unitary (i.e., one-piece) septa gasket 4002;

FIG. 40B is a side view of the port fitting 3608(3) of FIG. 40A without the septa gasket 4002;

FIG. 40C is a side view of the port fitting 3608(3) of FIG. 40B rotated 90 degrees about the vertical axis;

FIG. 41A is a perspective view of the septa gasket 4002 of FIG. 40A;

FIG. 41B is a cutaway, perspective view of the septa gasket 4002 of FIG. 40A;

FIG. 42A is an exploded, cut-away view of the port fitting 3608(3) of FIG. 36, the septa gasket 4002, and the canoe port 3612(3);

FIG. 42B is a cut-away view of the port fitting 3608(3) configured with the septa gasket 4002 and partially inserted into the canoe port 3612(3);

FIG. 42C is a cut-away view of the port fitting 3608(3) configured with the septa gasket 4002 and fully mounted within the canoe port 3612(3);

FIG. 42D shows a zoomed-in, cross-sectional view of a port fitting 3608 fully mounted within a canoe port 3612 of the canoe 3604 of FIG. 36;

FIG. 43A is a side view of an alternative, fixed port fitting 4302 with a tube 4312 inserted through a tube retainer 4308;

FIG. 43B is a side view of the port fitting 4302 with the tube 4312 rammed over the end of the tube barb 4304 before the tube retainer 4308 is rotated onto the threading 4306;

FIG. 43C is a side view of the port fitting 4302 after the tube retainer 4308 has been rotated onto the threading 4306;

FIG. 44A is a perspective view of an assembly that includes an alternative port sizer 4402 and a valve connector 4404;

FIG. 44B is a perspective view of the port sizer 4402 and the valve connector 4404;

FIG. 44C is a cutaway, perspective view of the assembly of FIG. 44A in its fully closed configuration;

FIG. 44D is a cutaway, perspective view of the assembly of FIG. 44A in an opened configuration;

FIG. 45 is a side view of the port sizer 4402;

FIG. 46 is a side view of the valve connector 4404;

FIG. 47 is a side view of the sealing gasket 4406 for the valve connector 4404;

FIG. 48 is a side view of the wiper ring 4408 for the valve connector 4404;

FIG. 49A is a cross-sectional side view of the assembly of FIG. 44C mounted within a canoe port 3612 and in the closed configuration;

FIG. 49B is a cross-sectional side view of the assembly of FIG. 44D mounted within a canoe port 3612 and in the opened configuration;

FIG. 50A is a perspective view of another assembly that includes another alternative port sizer 5002 and an alternative valve connector 5004, where the assembly is in its fully closed configuration;

FIG. 50B is a perspective view of the assembly of FIG. 50A, but in its fully opened configuration;

FIG. 50C is a cutaway, perspective view of the closed assembly of FIG. 50A;

FIG. 50D is a cutaway, perspective view of the opened assembly of FIG. 50B;

FIG. 51A is a side view of yet another assembly that includes yet another alternative port sizer 5102 and another alternative valve connector 5104, where the assembly is in its fully closed configuration;

FIG. 51B is a perspective view of the assembly of FIG. 51A in its fully closed configuration;

FIG. 51C is a cutaway, perspective view of the closed assembly of FIGS. 51A and 51B;

FIG. 51D is a cutaway, perspective view of the assembly of FIGS. 51A-51C, but in an opened configuration;

FIG. 51E is a perspective view of the valve connector 5104 of FIGS. 51A-51D configured with its sealing gasket 5106 and its wiper ring 5108;

FIG. 51F is an exploded view of the assembly of FIGS. 51A-51E;

FIG. 52A is a cross-sectional view of a bag assembly 5202;

FIG. 52B is a side view of the port fitting 5208 of FIG. 52A; and

FIG. 53 is a side view of an alternative port fitting 5302.

### DETAILED DESCRIPTION

Detailed illustrative embodiments of the present disclosure are disclosed herein. However, specific structural and functional details disclosed herein are merely representative for purposes of describing example embodiments of the present disclosure. The present disclosure may be embodied in many alternate forms and should not be construed as limited to only the embodiments set forth herein. Further, the terminology used herein is for the purpose of describing particular embodiments only and is not intended to be limiting of example embodiments of the disclosure.

As used herein, the singular forms “a,” “an,” and “the,” are intended to include the plural forms as well, unless the context clearly indicates otherwise. It further will be understood that the terms “comprises,” “comprising,” “contains,” “containing,” “includes,” and/or “including,” specify the presence of stated features, steps, or components, but do not preclude the presence or addition of one or more other features, steps, or components. It also should be noted that in some alternative implementations, the functions/acts noted may occur out of the order noted in the figures. For example, two figures shown in succession may in fact be executed substantially concurrently or may sometimes be executed in the reverse order, depending upon the functions/acts involved.

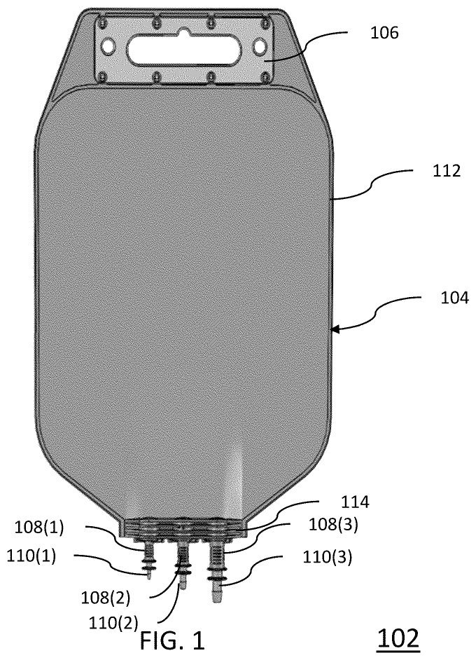

FIG. 1 is a plan view of a bag assembly 102 according to one embodiment of the present disclosure. The bag assembly 102 includes bag 104, bag hangar 106, three port sizers 108(1)-108(3), and three valve connectors 110(1)-110(3), where the bag 104 includes flexible bag chamber 112, bag hangar 106, and three-port canoe 114. Although the bag assembly 102 has a bag 104 of a certain size and a canoe 114 with three ports, those skilled in the art will understand that alternative embodiments of the present disclosure may have bags or different sizes and/or canoes having fewer or more ports.

As used herein, the term “port sizer” refers to a type of port fitting that receives a valve connector that supports both (i) a “closed” configuration in which the valve connector is inserted into the port sizer to a “closed position” at which fluid cannot flow through the port sizer and (ii) an “open” configuration in which the valve connector is inserted into the port sizer to an “open position” at which fluid can flow through the port sizer. Another type of port fitting, referred to herein as a “fixed port fitting,” is permanently in an open configuration and is not designed to receive a valve connector.

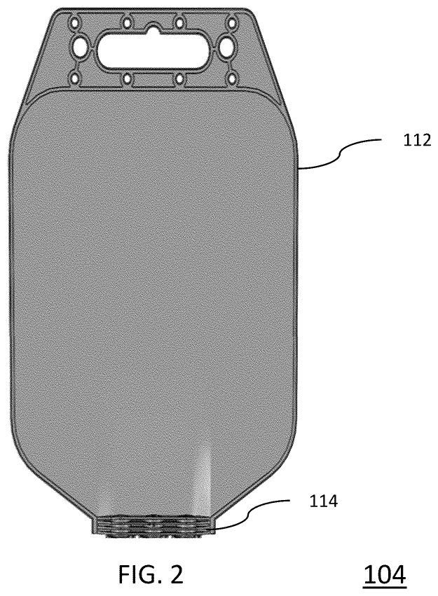

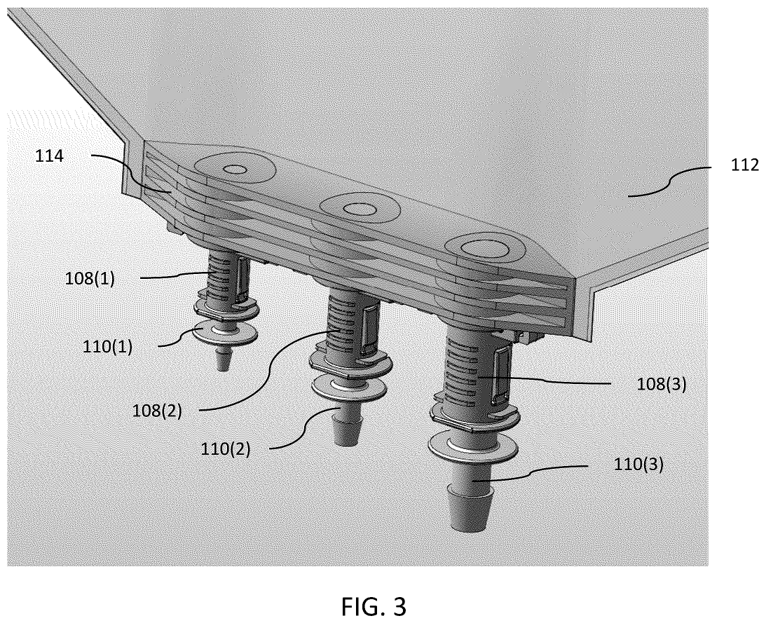

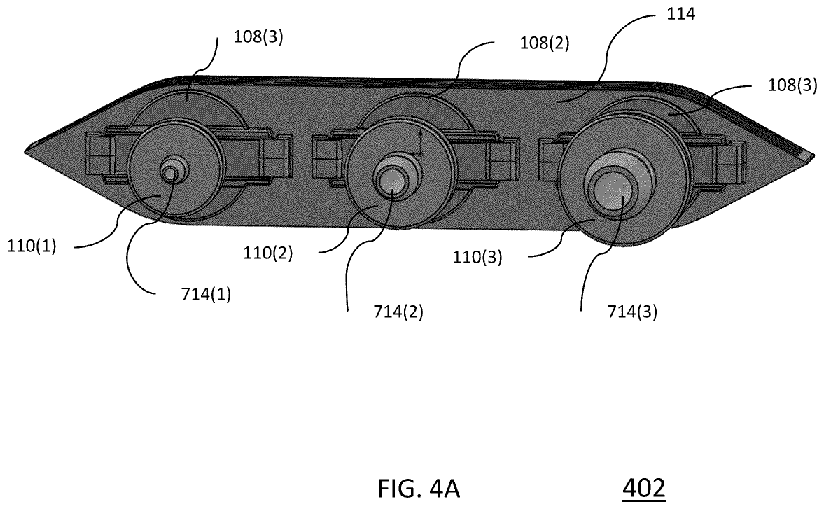

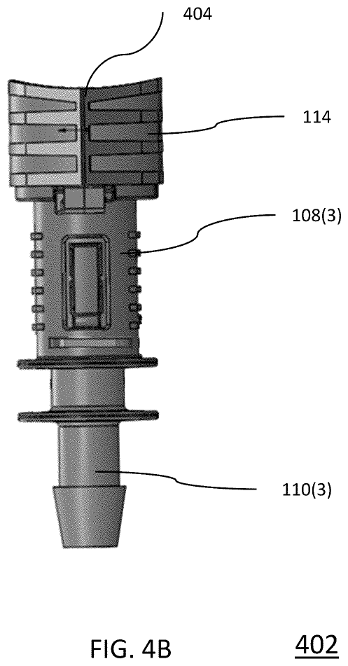

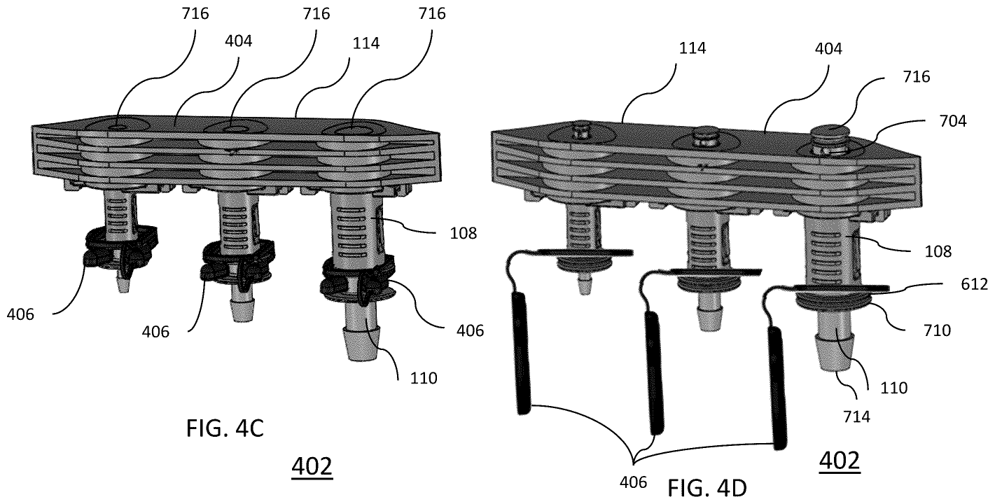

FIG. 2 is a plan view of the bag 104 of FIG. 1 without the bag hangar 106 and without any port sizers/fittings inserted into the canoe 114. FIG. 3 is an enlarged, perspective, X-ray view of the bottom of the bag assembly 102 of FIG. 1 showing the canoe 114, the three port sizers 108(1)-108(3), and the three valve connectors 110(1)-110(3). FIG. 4A is a perspective, bottom-up view of the canoe assembly 402 of FIG. 1 comprising the canoe 114, the three port sizers 108(1)-108(3), and the three valve connectors 110(1)-110(3). FIG. 4B is an end view of the canoe assembly 402 of FIG. 4A showing the port sizer 108(3) locked into the canoe 114, and the valve connector 110(3) inserted into, but not fully engaged with, the port sizer 108(3).

As known in the art, two pieces of a single-layer or multi-layer flexible plastic, fluoropolymer, or silicone film or some combination are assembled, e.g., by welding, to form the bag chamber 112. A single-port or multi-port, rigid, plastic or fluoropolymer, injection-molded canoe, such as the three-port canoe 114, is then assembled into an unwelded end of the bag chamber 112, e.g., by welding, to form a bag, such as the bag 104. Canoes of the present disclosure may be made of other suitable materials, such as (without limitation) thermoplastic elastomers (TPEs), thermoplastic polyurethane (TPU), or fluoropolymers, and/or manufactured using other suitable techniques, such as (without limitation) 3D printing.

The particular bag assembly 102 of FIG. 1 is configured with three differently sized pairs of port sizers 108 and valve connectors 110 sized to receive tubes (not shown) of three different sizes (e.g., having three different inner diameters). In particular, the bag assembly 102 has:
Small port sizer 108(1) and small valve connector 110(1) sized to receive a tube having, e.g., a ⅛-inch inner diameter;
Medium port sizer 108(2) and medium valve connector 110(2) sized to receive a tube having, e.g., a ¼-inch inner diameter; and
Large port sizer 108(3) and large valve connector 110(3) sized to receive a tube having, e.g., a ⅜-inch inner diameter.
   This provides the user the opportunity to select and customize the internal diameter of the fluid flow and thus the tube size. Those skilled in the art will understand that these sizes are examples and that other suitable sizes are possible.

As shown in FIG. 4A and as described in further detail below, the small, medium, and large valve connectors 110(1)-110(3) have small, medium, and large channels 714(1)-714(3), respectively, that enable different rates of fluid to flow between the interior of the bag 104 and the tubes in either direction. In other bag assemblies of the present disclosure, the bag 104 may be configured with any combination of three differently sized or same sized pairs of port sizers 108 and valve connectors 110.

As shown in FIG. 4B, the top surface 404 of the canoe 114 has a convex, groove-shaped curvature that provides a relatively smooth transition between the inner wall of the bag chamber 112 and the canoe top surface 404 that facilitates the removal of most if not all of the fluid in the bag 104 and/or eliminating fluid retain. Bioprocess fluids are typically expensive and any fluid that undesirably remains in the bag 104 may have significant value.

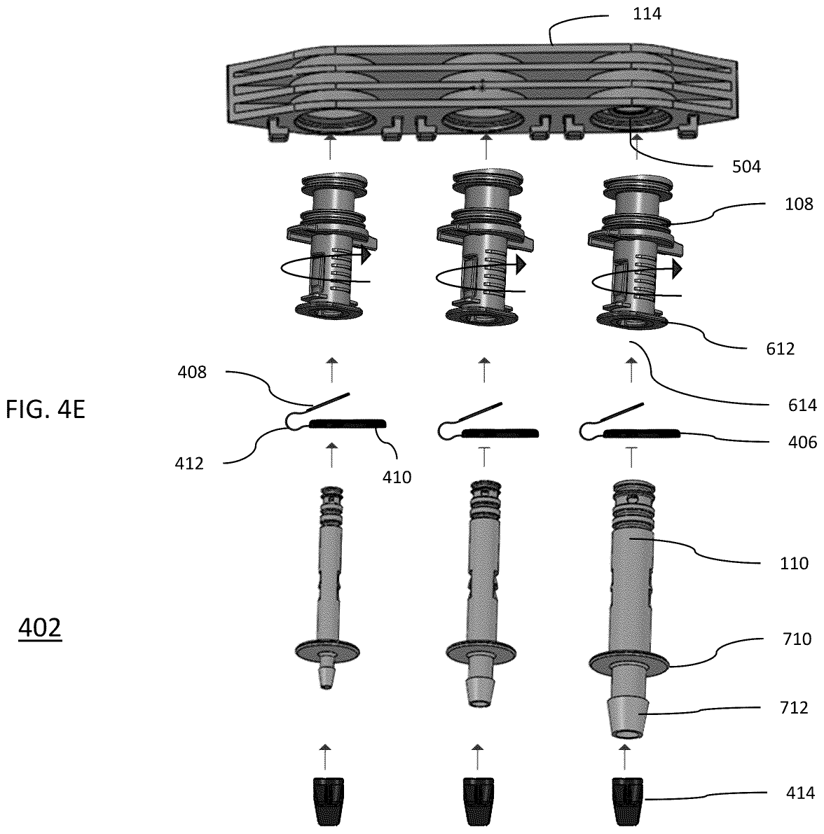

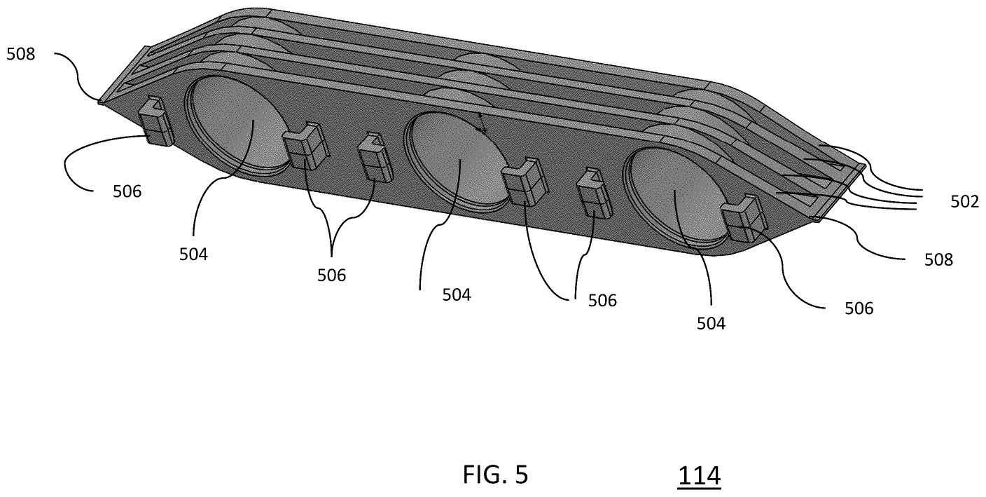

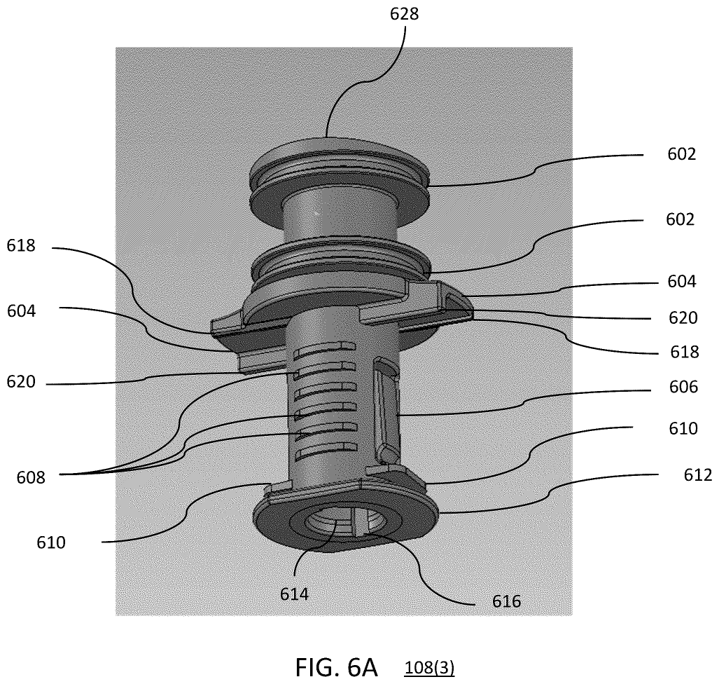

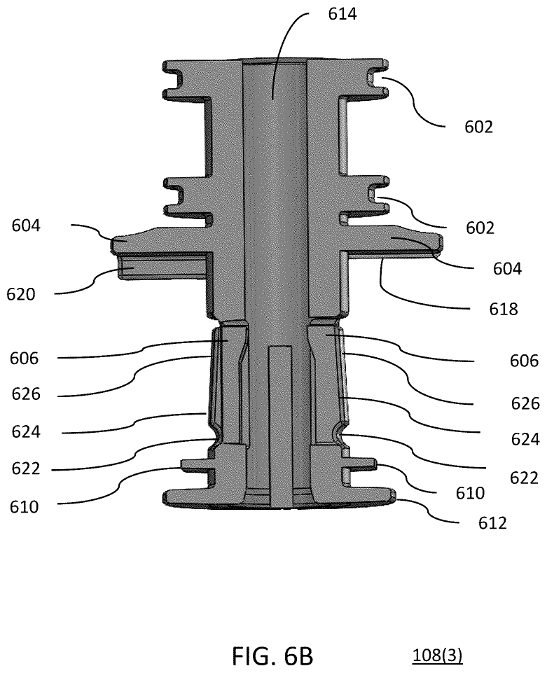

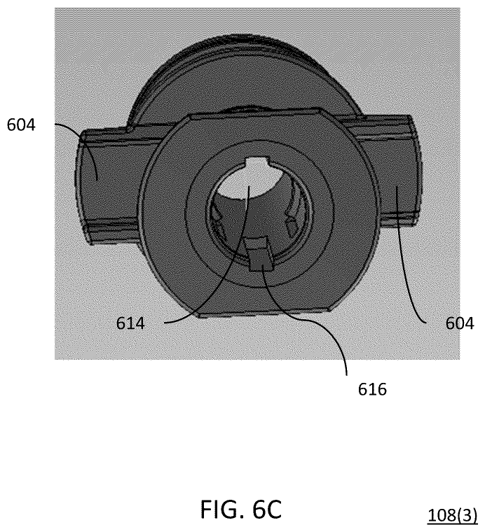

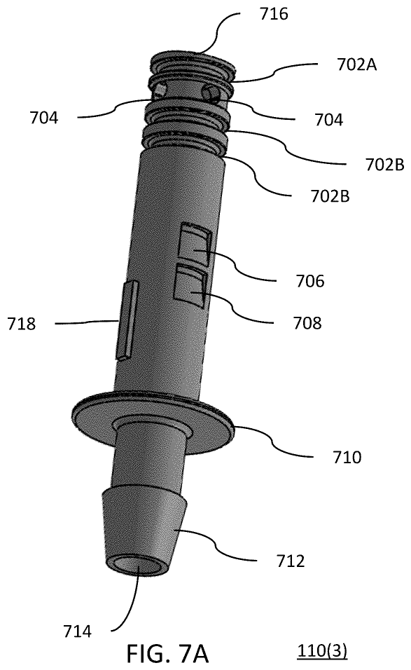

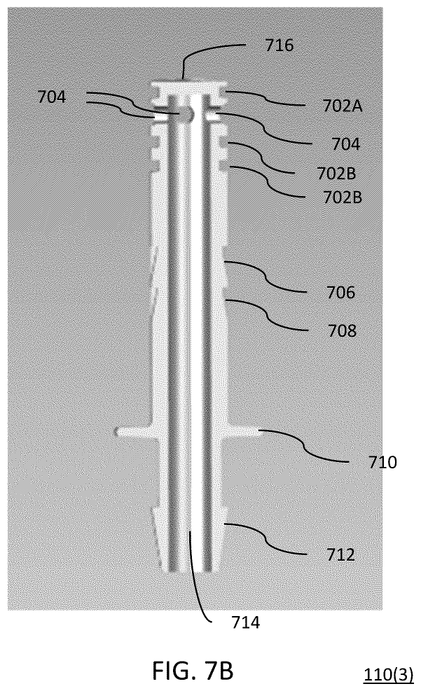

FIG. 4C is a perspective view of the canoe assembly 402 with the three valve connectors 110(1)-110(3) maintained at their “closed” configurations by three tethered safety clips 406. FIG. 4D is a perspective view of the canoe assembly 402 with the three tethered safety clips 406 disengaged and with the three valve connectors 110(1)-110(3) at their “open” configurations. FIG. 4E is an exploded, side view of the canoe assembly 402 of FIG. 4C. FIG. 5 is a perspective view showing the bottom of the canoe 114. FIG. 6A is a perspective view of the large port sizer 108(3). FIG. 6B is a cross-sectional side view of the large port sizer 108(3). FIG. 6C is a perspective bottom view of the large port sizer 108(3). FIG. 7A is a perspective view of the large valve connector 110(3). FIG. 7B is a cross-sectional side view of the large valve connector 110(3).

As shown in FIG. 5, the canoe 114 has (e.g., four) weld bars 502 that provide surfaces for welding the canoe 114 to the bag chamber 112 (FIG. 1) and three equally sized cylindrical through-holes (i.e., ports) 504, each of which has a corresponding pair of retention clips 506. Canoe 114 has tapered ends 508, which provide a sloping angle transition for welding the film of the bag chamber 112 against the canoe 114, thereby reducing the chance leak points to be created.

As shown in FIGS. 6A-6C for the large port sizer 108(3), each (e.g., rigid thermoplastic or fluoropolymer molded) port sizer 108 has two O-ring grooves 602 for receiving two respective (e.g., silicone or thermoplastic elastomer) O rings (not shown) to prevent leakage, two opposing locking arms 604, two opposing locking clips 606, a number of anti-slip grips 608, two tether tabs 610, a stop 612, and a cylindrical channel 614 running through the port sizer 108 from top to bottom. As explained further below, the bottom end of the port sizer channel 614 has a keyway 616. Note that the upper portions of the three differently sized port sizers 108(1)-108(3) of FIG. 1 have the same outer diameters (to enable any size port sizer 108 to be mounted into any of the three equally sized canoe through-holes 504), while the outer diameters of their lower portions are differently sized.

Each locking arm 604 has a detent 618 and a rotation stop 620. As shown in FIG. 6A and as described further below, the detents 618 and rotation stops 620 are configured on opposing sides of the locking arms 604 to enable the locking arms to engage the two corresponding canoe retention clips 506 when the port sizer 108 is permanently mounted into a canoe through-hole 504.

Port sizer 108(3) has two opposing tether tabs 610, which, as described further below, assist in retaining the tethered safety clip 406.

As shown most clearly in FIG. 6B, each port sizer locking clip 606 is a cantilevered structure that is connected to the rest of the port sizer 108 at only the bottom of the locking clip 606 at a relatively thin connection 622 that enables the cantilevered structure to rotate relative to that connection point. As described further below, the lower portion 624 of the locking clip 606 has a vertical inner surface, while the upper portion 626 has a beveled inner surface.

As shown in FIGS. 7A and 7B for the large valve connector 110(3), each (e.g., rigid plastic or fluoropolymer molded or 3D printed) valve connector 110 has one upper O-ring groove 702A and two lower O-ring grooves 702B for receiving three respective (e.g., silicone or thermoplastic elastomer) O rings (not shown) to prevent leakage, one or more (in this embodiment, four) side ports 704, two opposing, “closed” locking tabs 706 (only one of which is visible in FIG. 7A), two opposing, “open” locking tabs 708 (only one of which is visible in FIG. 7A), a stop 710, a tube barb 712, and a channel 714 running through the valve connector 110 from the side ports 704 at the top to the open bottom that allows fluid to flow into or out of the bag chamber 112 through the valve connector 110. Note that the top surface 716 of the valve connector 110 has a convex shape that matches the convex shape of the canoe top surface 404 (FIG. 4B) and the convex shape of the top 628 of the port sizer (as shown in FIG. 6A) to form a smooth curvilinear surface when the port sizer is mounted onto the canoe and the valve connector is inserted into the port sizer up to the closed position.

Note that the numbers of O-ring grooves in the port sizers 108 and/or in the valve connectors 110 may be different in different embodiments of the present disclosure from those shown in the figures.

As shown in FIGS. 4C and 4E and as described further below, each (e.g., plastic, TPU, TPE, EPDM, rubber, silicone, elastomeric material, or fluoropolymer molded) tethered safety clip 406 has a (e.g., semi-rigid) tether ring 408 and a (e.g., relatively rigid) tether clip 410 interconnected by a (e.g., flexible) tether 412. Since these elements of the tethered safety clip 406 are formed of the same material, the relative rigidity/flexibility of these elements is a function of their relative thicknesses.

As shown in the “closed” configuration of FIG. 4C, the convex top surface 716 of each valve connector 110 is flush with the convex canoe top surface 404 with the corresponding tethered safety clip 406 preventing the valve connector 110 from transitioning from the “closed” configuration of FIG. 4C to the “open” configuration of FIG. 4D.

As shown in the “open configuration of FIG. 4D, after removing each tethered safety clip 406, the corresponding valve connector 110 can be pushed further into the port sizer 108 until the valve connector stop 710 abuts the port sizer stop 612, at which position, the valve connector top surface 716 extends above the canoe top surface 404, thereby exposing the valve connector side ports 704 and enabling fluid to flow between the interior of the bag 104 and the valve connector channel 714.

As represented in FIG. 4E and as described further below, the canoe assembly 402 of FIG. 4C may be assembled by:
Placing a dust cap 414 (not shown in FIG. 4C) over the barbed end of the tube barb 712 of each valve connector 110;
Looping the tether ring 408 of each tethered safety clip 406 around and over the port sizer stop 612 at the bottom of the corresponding port sizer 108;
Inserting each valve connector 110 (configured with three O-rings) into the channel 614 of the corresponding port sizer 108;
Attaching the tether clip 410 of each tethered safety clip 406 to the corresponding valve connector 110 just above the valve connector stop 710 and just below the port sizer stop 612; and
Mounting each port sizer 108 (configured with two O-rings) into the corresponding canoe through-hole 504.
   Note that the canoe assembly 402 of FIG. 4C can be alternatively assembled by performing these steps in different appropriate sequences.

To insert a valve connector 110 into a correspondingly sized port sizer 108, the top portion of the valve connector 110 (configured with three O-rings) is inserted into the bottom end of the port sizer 108 with the valve connector keyway guide 718 aligned to engage the port sizer keyway 616, which in turn aligns the valve connector locking tabs 706 and 708 with the port sizer locking clips 606.

When the valve connector 110 is initially inserted into the port sizer 108 to the “closed” configuration, the port sizer locking clips 606 will engage with the valve connector “closed” locking tabs 706. In particular, as the valve connector 110 proceeds along the port sizer channel 614, the port sizer locking clips 606 will pivot outwardly about their locking clip connections 622 as the outer surface of valve connector 110 pushes outwardly on the beveled surfaces of the locking clips 606 until the “closed” configuration is reached, at which point, the beveled upper portions of the locking clips 606 will be received within the beveled valve connector “closed” locking tabs 706, which prevents subsequent removal of the valve connector 110 from the port sizer 108. In this “closed” configuration, the valve connector's uppermost O-ring is still within the port sizer channel 614 such that fluid flow into and out of the bag 104 through the valve connector 110 is prevented. In addition, in this “closed” configuration, the convex valve connector top surface 716 is flush with the convex canoe top surface 404 (FIG. 4B) to form a contiguous convex groove at the bottom of the bag 104. Through the use of a rotational thread pattern external to the valve connector (110) and a corresponding threaded groove on the internal diameter of the port sizer (108), the valve can be rotated up into an open position, allowing fluid flow. The same feature on the valve connector would then also allow the valve connector to be retracted into a closed position.

Note that the port sizer locking clips 606 and the valve connector “closed” locking tabs 706 are designed to prevent the valve connector 110 from being removed from the port sizer 108 after the “closed” configuration has been achieved. In alternative embodiments, the valve connectors and the port sizers may be designed such that the valve connectors are removable from the port sizers.

As described further below, after achieving the “closed” configuration and with the tether clip 410 of the tethered safety clip 406 disengaged from the valve connector 110, the “open” configuration can be achieved by pushing the valve connector 110 further into the port sizer 108 such that the port sizer locking clips 606 will transition from engaging with the valve connector “closed” locking tabs 706 to be engaged with the valve connector “open” locking tabs 708 in a manner analogous to the initial transition to the “closed” configuration. At this “open” configuration, the valve connector stop 710 abuts the port sizer stop 612, thereby preventing the valve connector 110 from being pushed any further into the port sizer 108 in order to prevent leakage.

Note that the port sizer locking clips 606 and the valve connector “open” locking tabs 708 are designed to prevent the valve connector 110 from being removed from the “open” configuration after the “open” configuration has been achieved. In alternative embodiments, the valve connectors and the port sizers may be designed (i) to enable the valve connectors to transition from the “open” configuration back to the “closed” configuration and (ii) possibly to enable the valve connectors to be removable from the port sizers.

To mount a port sizer 108 into a canoe through-hole 504, the top portion of the port sizer 108 (configured with two O-rings) is inserted into the canoe through-hole 504 until the port sizer locking arms 604 abut the bottom surface of the canoe 114 and then the inserted port sizer 108 is rotated clockwise to engage the two port sizer locking arms 604 with the two corresponding canoe retention clips 506 to keep the port sizer 108 in place within the canoe through-hole 504. Note that the port sizer locking arms 604 have rotation stops 620 that stop the clockwise rotation of the port sizer 108 with respect to the canoe 114 to retain the top curvature profile when the rotation stops 620 abut the sides of the canoe retention clips 506. Note further that the locking arms 604 has detents 618 that prevent the mounted port sizer 108 from being removed from the canoe 114. The main functions of the port sizers 108 are to determine the inside diameter of the fluid path and, in some instances, to house the function of the valve connectors 110. The valve connectors allow the operator to open the bag 104 for fluid input and removal.

Referring again to the bag assembly 102 of FIG. 1, as known in the art, up to three tubes (not shown) of different size can be connected and secured to the three valve connectors 110, for example, using tie wraps, Barblocks, Oetiker clamps, hose clamps, wires, or any other suitable tubing retaining device (not shown) wrapped around the tubes above the barbed ends of the tube barbs 712. The bag assembly 102 of FIG. 1 can then be used to move fluids into or out of the bag 104 at any combination of flow rates supported by the differently sized port sizer/valve connector/tube sub-assemblies by disengaging the tether clip 410 of each corresponding tethered safety clip 406 (if present) and moving each corresponding valve connector 110 from its “closed” configurations to its “open” configuration.

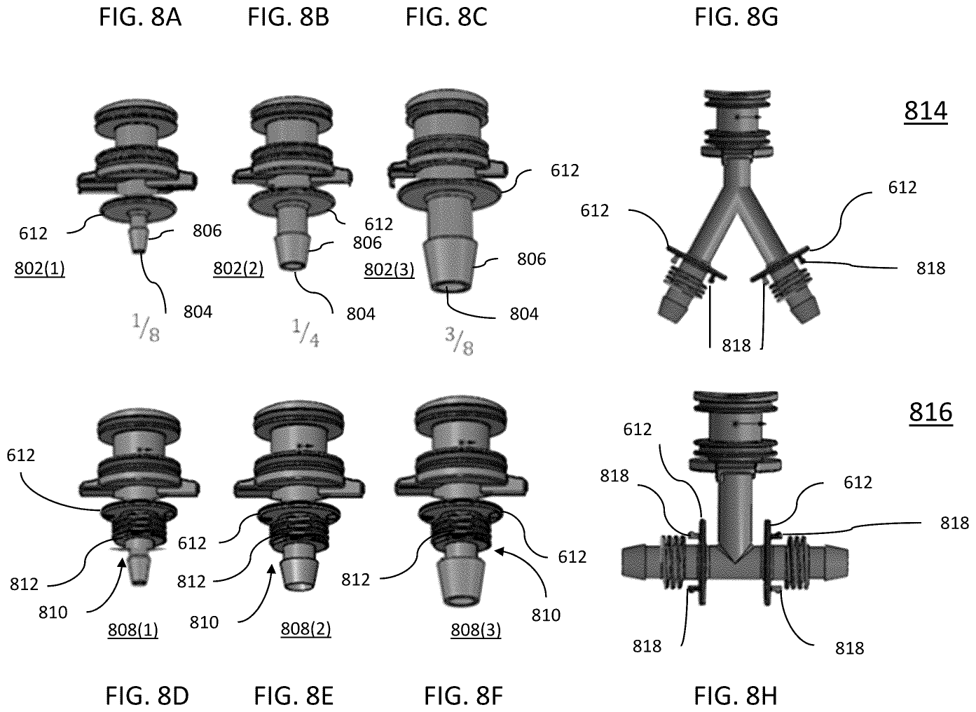

FIGS. 8A-8C show perspective views of small, medium, and large, unthreaded, fixed port fittings 802(1)-802(3), respectively, each of which can be used instead of a corresponding port sizer 108/valve connectors 110 pair. Each fixed port fitting 802 is a unitary structure having a single, fixed “open” configuration and a channel 804 that runs entirely through the fixed port fitting 802 from top to bottom. Each fixed port fitting 802 has (i) two O-ring grooves and two opposing locking arms that are analogous to the corresponding elements of the corresponding port sizer 108 and (ii) a stop 612 and a tube barb 806 that are analogous to the corresponding elements of the corresponding valve connector 110. Since they are always in their “open” configuration, fixed port fittings 802 can be used with conventional tube clamps to control the flow of fluid into and out of the bag 104.

FIGS. 8D-8F show perspective views of small, medium, and large, threaded, fixed port fittings 808 having threaded tube barbs 810 with threading 812. Otherwise, the threaded, fixed port fittings 808 are similar to the corresponding “unthreaded,” fixed port fittings 802 of FIGS. 8A-8C.

FIG. 8G shows a side view of a fixed port fitting 814 having an integral, threaded “wye” tube connector with threaded tube barbs, and FIG. 8H shows a side view of a fixed port fitting 816 having an integral, threaded “tee” tube connector with threaded tube barbs. As shown in FIGS. 8G and 8H and as described further below, each port fitting stop 612 has two opposing retainer locking tabs 818.

FIG. 8I shows a perspective view of a fixed port fitting 802A according to an alternative embodiment. As shown in FIG. 8I, port fitting 802A has extended locking arms 803 (only one of which is seen in FIG. 8I) that extend to the stop 612 that make it easier to rotate the port fitting 802A into a canoe 114, thereby improving the operator's ability to engage the locking mechanism of the port fitting 802A. Analogous extended locking arms can be incorporated into other port fittings of the present disclosure.

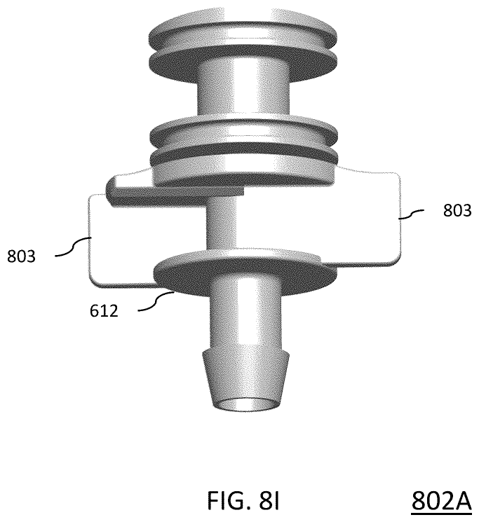

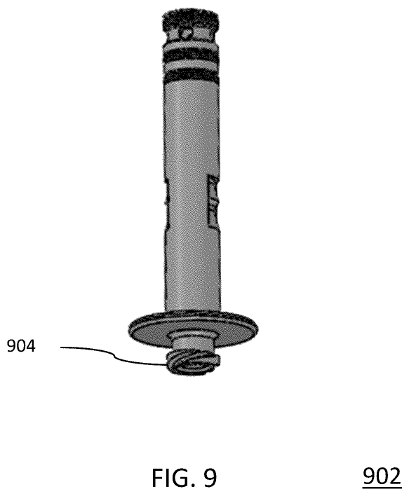

FIG. 9 is a perspective view of a luer lock valve connector 902 having a luer lock 904 instead of a tube barb for mating with a tube (not shown).

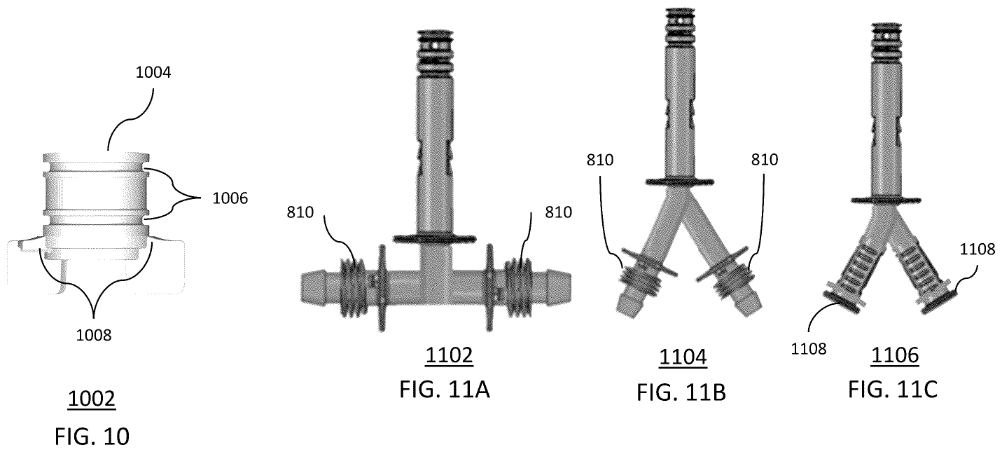

FIG. 10 is a block-out plug 1002 that can be used instead of a port sizer 108/valve connector 110 combination or a fixed port fitting 802/808 to permanently close a canoe through-hole 504 to effectively reduce the number of operable ports in the canoe, if needed. The block-out plug 1002 has a curved top surface 1004, O-ring grooves 1006, and locking arms 1008 analogous to the locking arms 604 of the port fittings 108, 802, and 808.

FIGS. 11A-11C respectively show a “tee” valve connector 1102 having two threaded tube barbs 810, a “wye” valve connector 1104 having two threaded tube barbs 810, and a “wye” valve connector 1106 having two valve-connector housings 1108, where each valve-connector housing 1108 can receive a valve connector (not shown). Each of these three connectors 1102-1106 can be configured with a corresponding port sizer 108 in either a “closed” configuration or an “open” configuration analogous to those described previously.

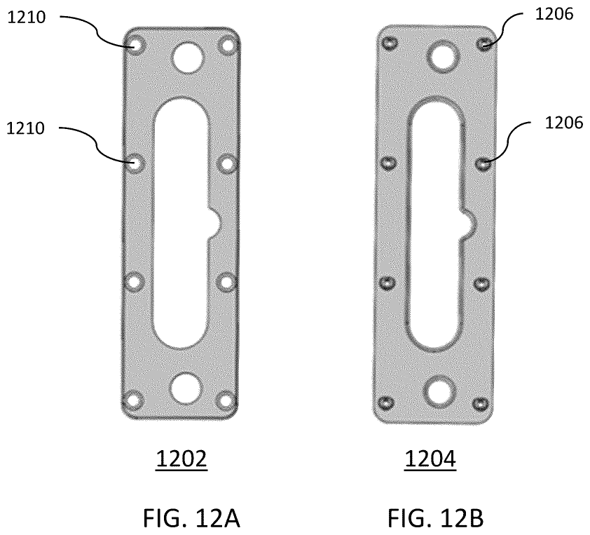

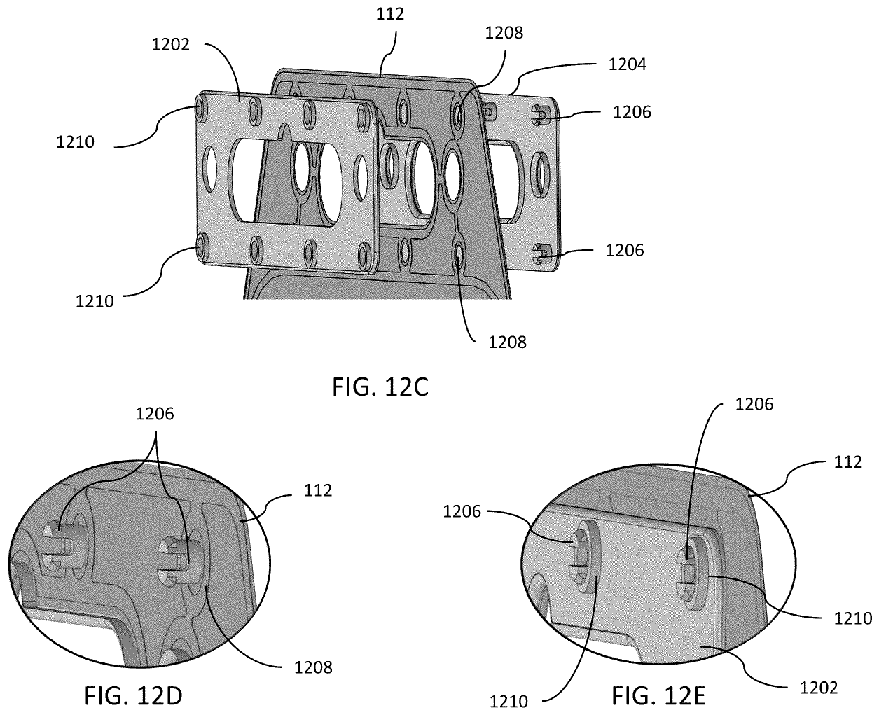

FIGS. 12A and 12B are plan views of female and male bag hangar components 1202 and 1204, respectively, which can be used to form the bag hanger 106 of FIG. 1. FIG. 12C shows a perspective view of the female and male bag hangar components 1202 and 1204 positioned to be configured to the top portion of a bag chamber 112, where the top portion is outside of the bag enclosure that holds fluid. FIG. 12D shows a zoomed-in, perspective view of the male bag hangar component 1204 configured with the bag chamber 112, while FIG. 12E shows a zoomed-in perspective view of the final bag hangar assembly. As shown in FIGS. 12A-12E, the male bag hanger component 1204 has barbed male elements 1206 that are pushed through corresponding openings 1208 in the bag chamber 112 and then through corresponding female elements 1210 in the female bag hangar component 1202 to form and secure the bag hangar 106 (FIG. 1) onto the bag chamber 112. Those skilled in the art will understand that the bag hangar 106 is used to reinforce larger bags 104 from the full weight of the liquid contents.

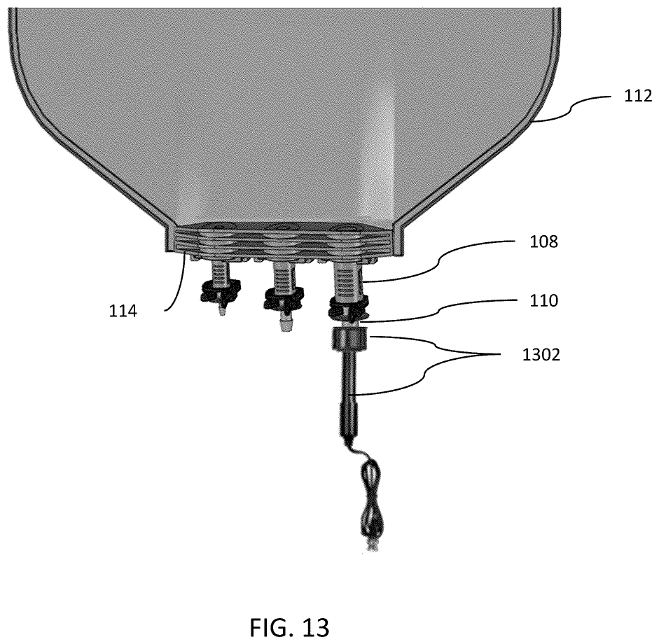

FIG. 13 shows a side view of the bag assembly 102 of FIG. 1 with a bag probe 1302 attached to one of the valve connectors 110. Depending on the configuration, the bag probe 1302 can be used to measure any suitable characteristic(s) of fluid retained in the bag, such as (without limitation) temperature, pH, salinity, dissolved oxygen level, viable cell density, culture viability, growth rate, and/or nutrient levels. Another version includes the probe assembled directly into the port sizer without the need to move the probe in or out of the bag.

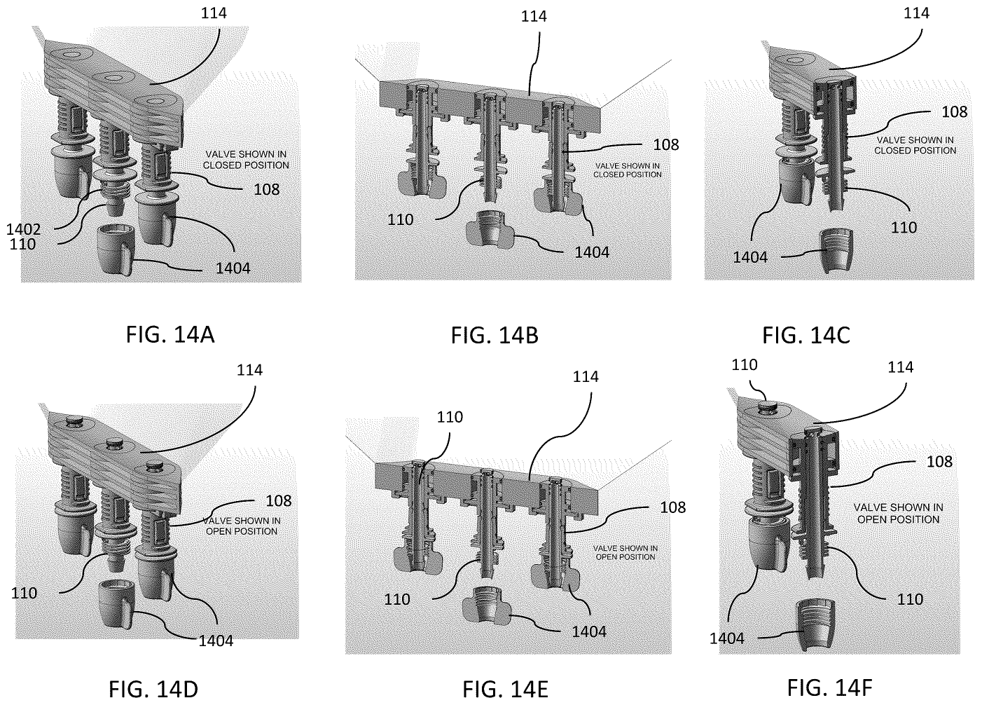

FIG. 14A is a perspective view of a canoe assembly 1400 having a tube retainer design in its “closed” configuration where (i) the valve connectors 110 have threaded tube barbs 810 with threading 1402 and (ii) threaded tube retainers 1404 engage with the threaded tube barbs 810 to assist in sealing and retaining a tube (not shown) onto the barbed end of the threaded tube barb 810. FIG. 14B is a cross-sectional perspective side view of the canoe assembly 1400 of FIG. 14A, and FIG. 14C is a cross-sectional perspective end view of the canoe assembly 1400 of FIG. 14A.

FIGS. 14D-14F are analogous to the views of FIGS. 14A-14C, but with the canoe assembly 1400 in its “open” configuration with the ends of the valve connectors 110 extending above the top surface of the canoe 114. Note that the tubes are not shown in FIGS. 14A-14F.

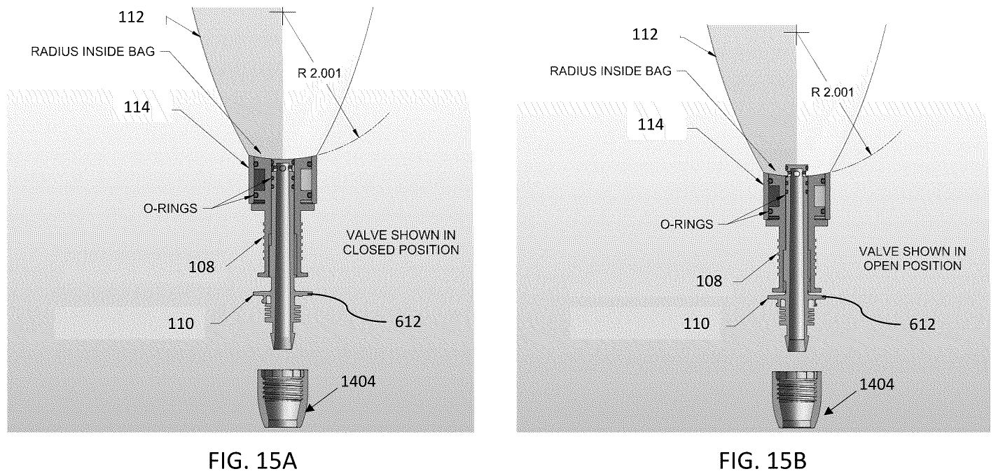

FIG. 15A is a cross-sectional end view of the canoe assembly 1400 of FIG. 14A in its “closed” configuration, and FIG. 15B is a cross-sectional end view of the canoe assembly 1400 of FIG. 14D in its “open” configuration.

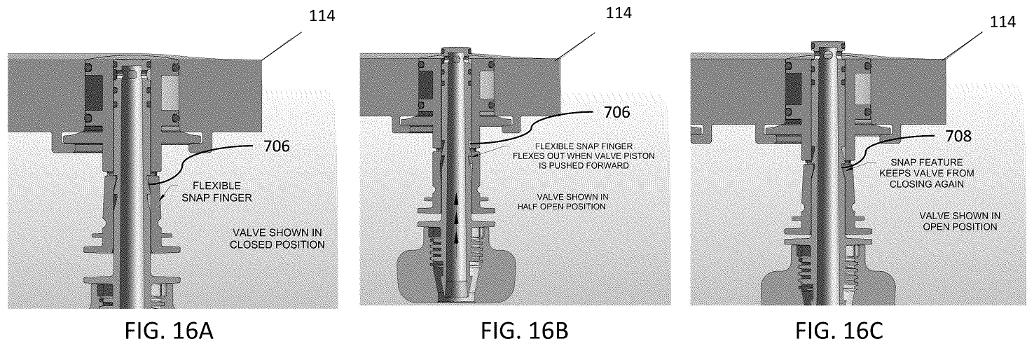

FIG. 16A is a zoomed-in, cross-sectional side view of the canoe assembly 1400 of FIG. 14A with the port sizer locking clips 606 engaged with the valve connector “closed” locking tabs 706. FIG. 16C is a zoomed-in, cross-sectional side view of the canoe assembly 1400 of FIG. 14D with the port sizer locking clips 606 engaged with the valve connector “open” locking tabs 708. FIG. 16B is a zoomed-in, cross-sectional side view of the same canoe assembly 1400 transitioning from the “closed” configuration of FIG. 14A to the “open” configuration of FIG. 14D, during which, the port sizer locking clips 606 flex outward due to the beveled outer diameter of the valve connector “closed” locking tabs 706. Note that the tubes are not shown in FIGS. 16A-16C.

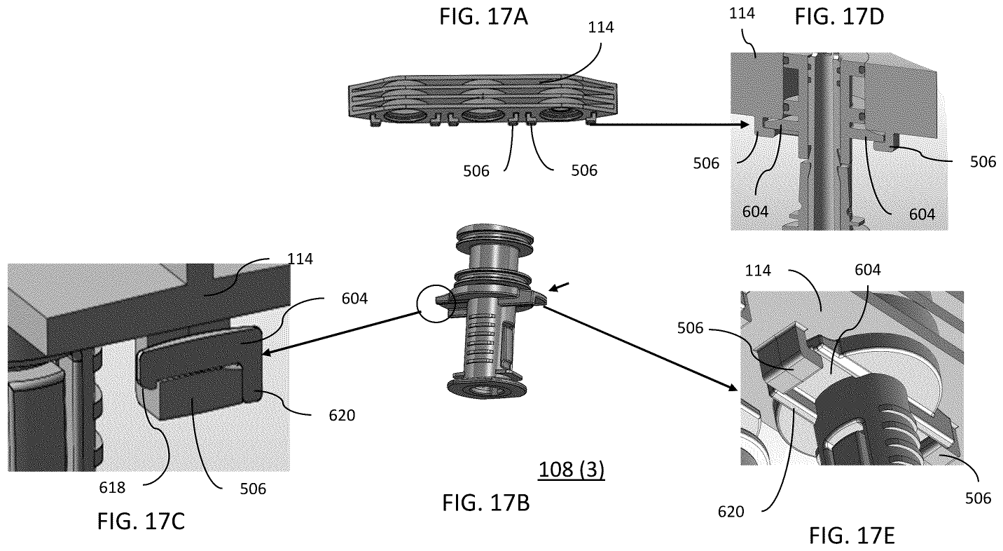

FIGS. 17A and 17B show perspective views of the canoe 114 and the port sizer 108(3), respectively. FIGS. 17C and 17D show different, zoomed-in, cutaway, perspective views, and FIG. 17E shows a zoomed-in, perspective view showing how the port sizer locking arms 604 with their rotation stops 620 and detents 618 engage with the canoe retention clips 506.

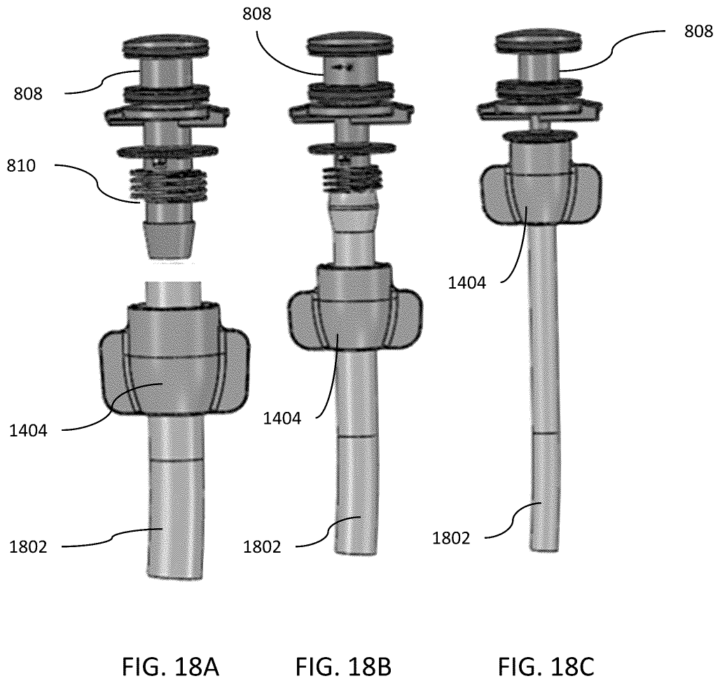

FIG. 18A shows a side view of a large tube (aka tubing) 1802 having a large tube retainer 1404 prior to a large tube 1802 being attached to the threaded tube barb 810 of a large, threaded, fixed port fitting 808. FIG. 18C shows a side view of a medium tube 1802 having a medium tube retainer 1404 after a medium tube 1802 has been attached to the threaded tube barb 810 of a medium, threaded, fixed port fitting 808, but before the tube retainer 1404 is connected to the threaded tube barb 810. FIG. 18C shows a side view of a small tube 1802 having a small tube retainer 1404 after a small tube 1802 has been attached to the threaded tube barb 810 of a small threaded fixed port fitting 808 and after the tube retainer 1404 has been connected to the threaded tube barb 810.

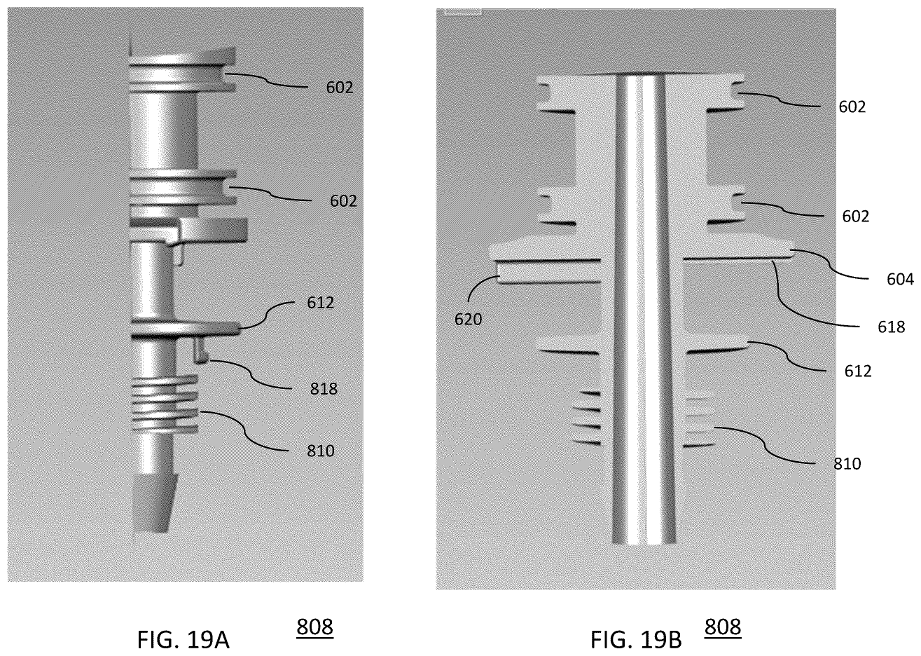

FIG. 19A is a side view of half of a threaded fixed port fitting 808, and FIG. 19B is a cross-sectional side view of the threaded fixed port fitting 808 of FIG. 19A.

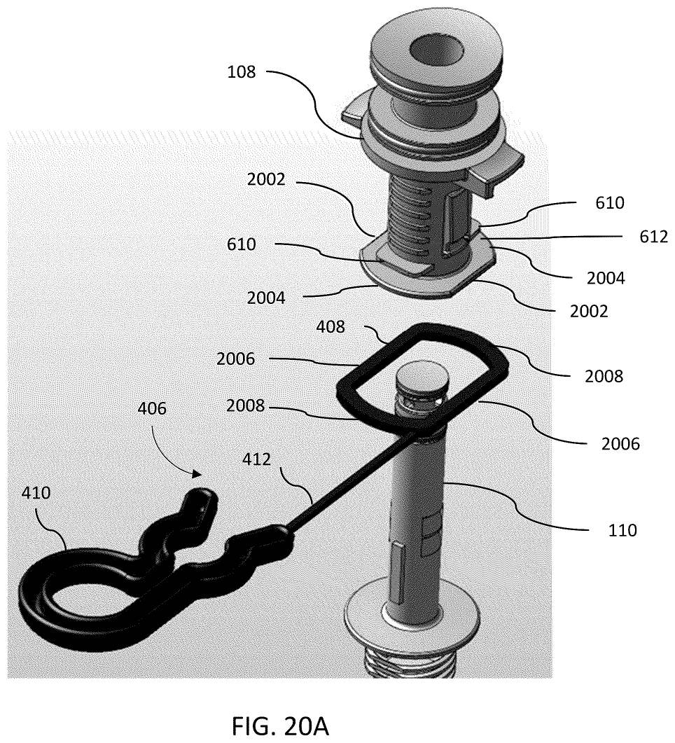

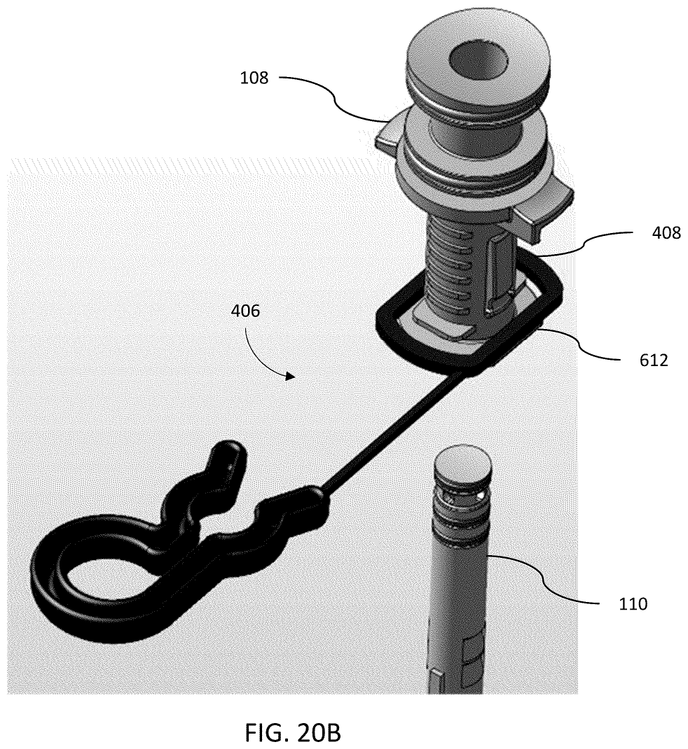

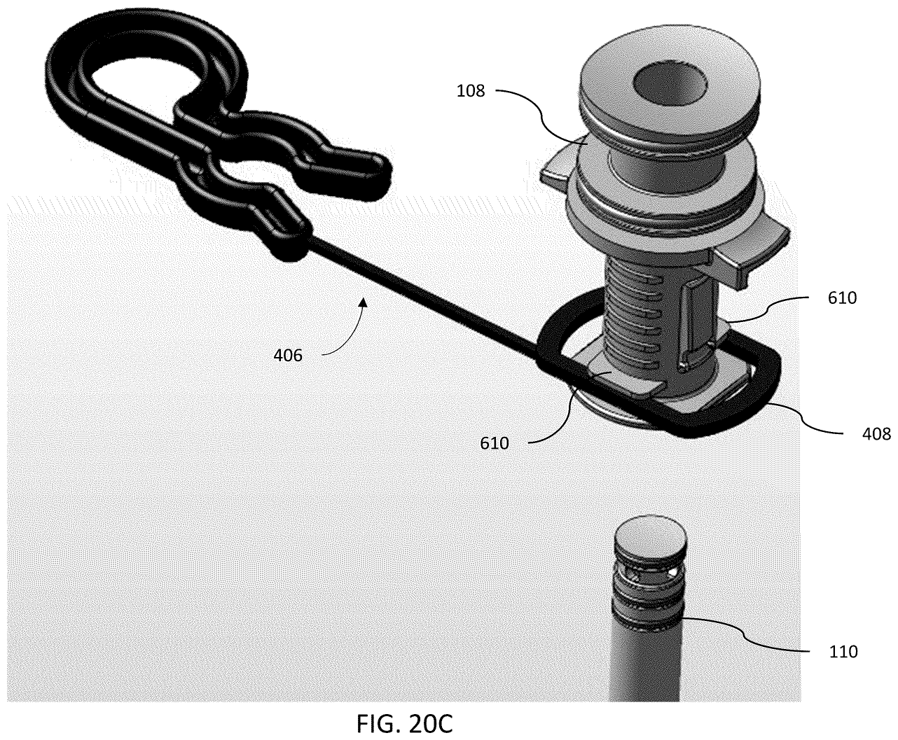

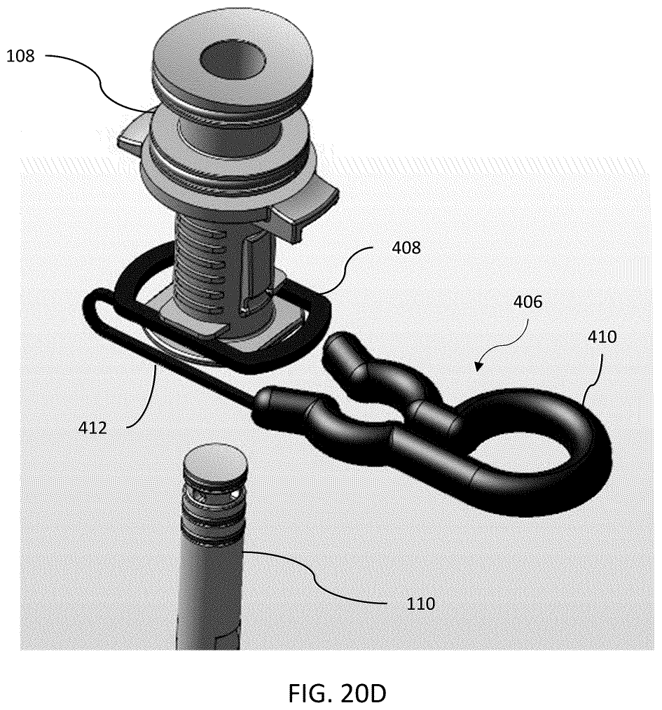

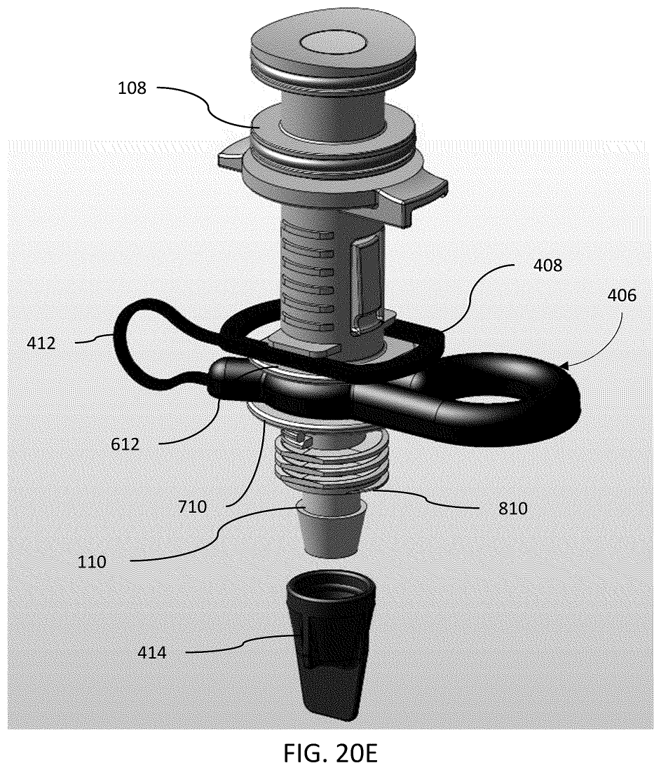

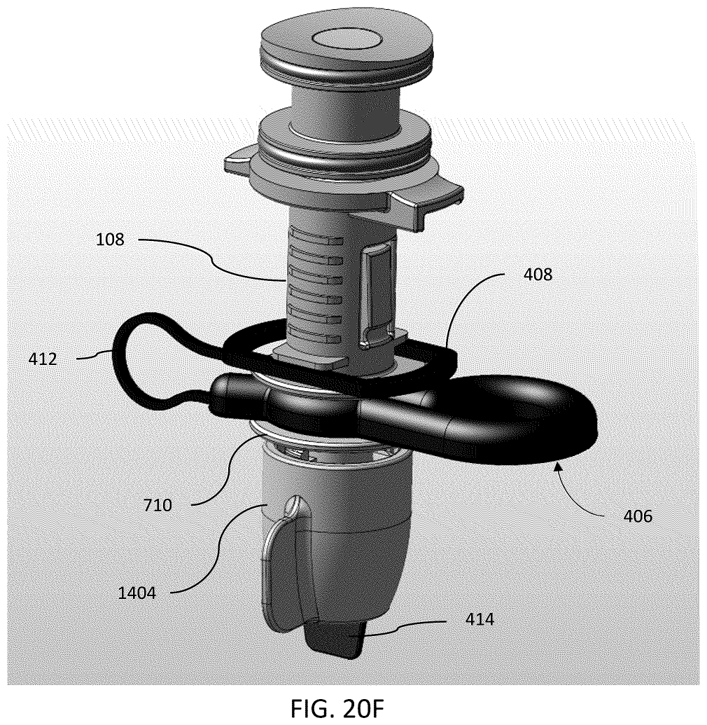

FIG. 20A shows a perspective view of a port sizer 108, a valve connector 110, and a tethered safety clip 406 before they are assembled together. As shown in FIG. 20A, the port sizer 108 has (i) a stop 612 having two opposing, straight edges 2002 and two opposing, curved edges 2004 and (ii) two tether tabs 610. In addition, the tethered safety clip 406 has a tether ring 408 and a tether clip 410 interconnected by a flexible tether 412, where the tether ring 408 has two opposing, straight portions 2006 and two opposing, curved portions 2008.

FIG. 20B shows a perspective view of the elements of FIG. 20A after the tether ring 408 has been placed over the port sizer stop 612. Note that the width of the tether ring 408 is slightly smaller than the width of the port sizer stop 612, but that the length of the tether ring 408 is slightly larger than the length of the port sizer stop 612, thereby enabling the port sizer stop 612 to be worked through the tether ring 408.

FIG. 20C shows a perspective view of the elements of FIG. 20B after the tether ring 408 has been rotated clockwise about 90 degrees with respect to the port sizer 108. Note that the thickness of the tether ring 408 is slightly greater than the distance between the top surface of the port sizer stop 612 and the bottom surfaces of the tether tabs 610 such that the tether ring 408 is held in place by the compressive force applied by the port sizer stop 612 and the tether tabs 610.

FIG. 20D shows a perspective view of the elements of FIG. 20C after the tether clip 410 has been rotated 180 degrees with respect to the tether ring 408, resulting in a bending of the tether 412.

FIG. 20E shows a perspective view of the elements of FIG. 20D and a dust cap 414 after (i) the valve connector 110 has been inserting into the port sizer 108 up to the “closed” configuration and (ii) the tether clip 410 has been pushed onto the valve connector 110 just below the port sizer stop 612 and just above the valve connector stop 710, thereby preventing the valve connector 110 from inadvertently being transitioned from the “closed” configuration to the “open” configuration. FIG. 20E shows the threaded tube barb 810 of the valve connector 110.

FIG. 20F shows a perspective view of the elements of FIG. 20E and a tube retainer 1404 after (i) the dust cap 414 has been placed over the barbed end of the threaded tube barb 810 (not visible in FIG. 20F) and (ii) a tube retainer 1404 has been screwed onto the threaded tube barb 810. The dust cap 414 is in place during pre-assembly, storage, and transportation. When the final assembly is completed, the dust cap will be removed and discarded.

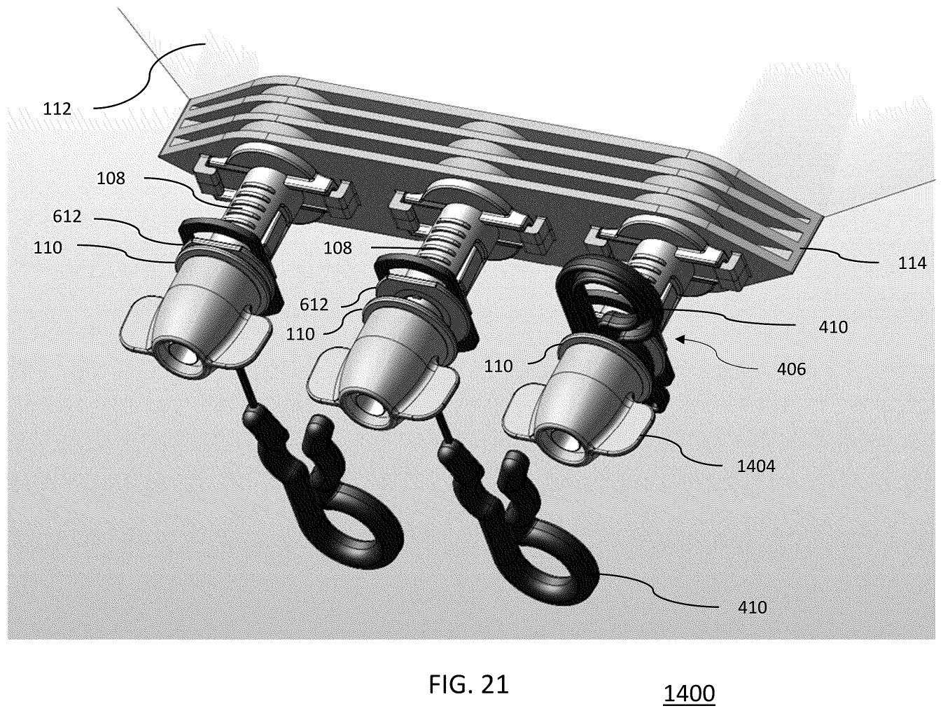

FIG. 21 shows a perspective view of the bottom of a canoe assembly 1400 having three port sizer/valve connector sub-assemblies in three different configurations. In the right-most sub-assembly, the valve connector 110 is in the “closed” configuration and the tether clip 410 is engaged on the valve connector 110, as in FIG. 20F except without the dust cap 414. In the middle sub-assembly, the tether clip 410 has been disengaged from the valve connector 110 with the valve connector 110 still in the “closed” configuration. In the left-most sub-assembly, the valve connector 110 has been moved to the “open” configuration with the valve connector stop 710 abutting the port sizer stop 612. Note that the tubes that would be connected to the valve connectors 110 and covered by the tube retainers 1404 are not shown in this view.

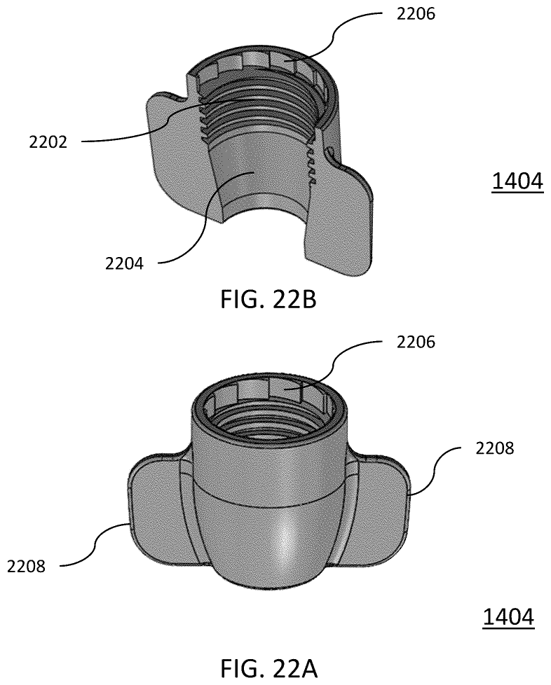

FIG. 22A is a perspective view of a tube retainer 1404. FIG. 22B is a cross-sectional perspective view of the tube retainer 1404 of FIG. 22A. As shown in FIGS. 22A and 22B, the tube retainer 1404 has a threaded cylindrical portion 2202 that engages with a threaded tube barb 810 and a smooth frustum portion 2204 that squeezes an intervening tube between the tube retainer 1404 and the barbed end of a threaded tube barb 810 to prevent leakage at the tube/tube barb interface. The tube retainer 1404 also has anti-rotation features 2206 configured to engage the retainer locking tabs 818 on the valve connector stop 710 of a threaded tube barb 810, thereby preventing the tube retainer 1404 from being removed from a threaded tube barb 810 once it has been engaged. The tube retainer 1404 has two opposing wings 2208 that provide leverage for rotating the tube retainer 1404 onto the threaded tube barb 810.

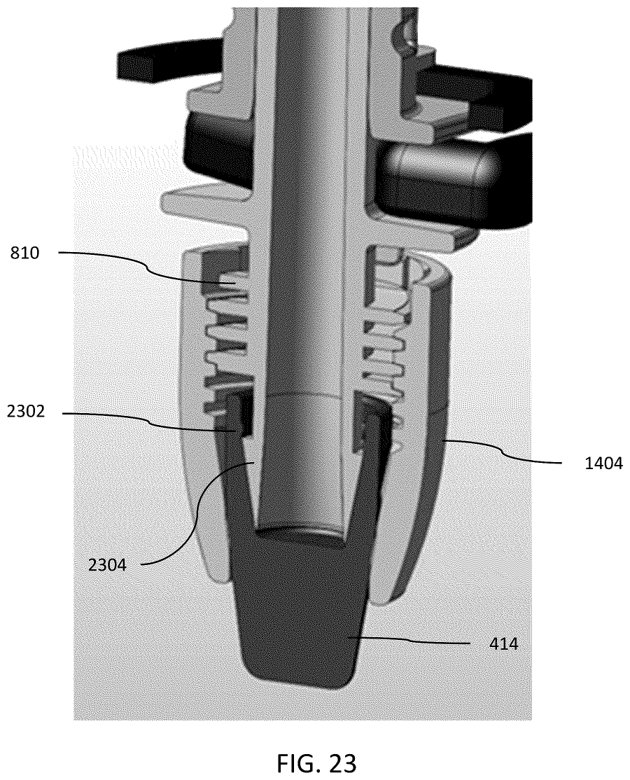

FIG. 23 is a zoomed-in cross-sectional perspective view of a tube retainer 1404 screwed onto a threaded tube barb 810 with an intervening dust cap 414. The dust cap 414 has a small detent feature 2302 that engages with the barbed end 2304 of the tube barb 810 to hold the dust cap 414 onto the tube barb 810, but allows the dust cap 414 to be removed from the tube barb 810. The dust cap 414 prevents the tube retainer 1404 from being fully screwed onto the threaded tube barb 810 and stops the tube retainer 1404 before the anti-rotation features 2206 on the tube retainer 1404 start to engage with the retainer locking tabs 818 of the threaded tube barb 810.

When a tube (not shown in FIG. 23) is to be connected to the threaded tube barb 810, the tube retainer 1404 is removed from the threaded tube barb to enable the dust cap 414 to be removed. At this point, the tube may be inserted through the bottom end of the tube retainer 1404 and onto the barbed end 2304 of the threaded tube barb 810. The tube retainer 1404 may then be fully screwed onto the threaded tube barb 810 such that the anti-rotation features 2206 on the tube retainer 1404 engage with the retainer locking tabs 818 of the threaded tube barb 810 to permanently secure and seal the tube onto the threaded tube barb 810. The design of the tube retainer 1404 accommodates different flexible tube wall thicknesses, effectively compressing the tubing to create a leak-proof seal.

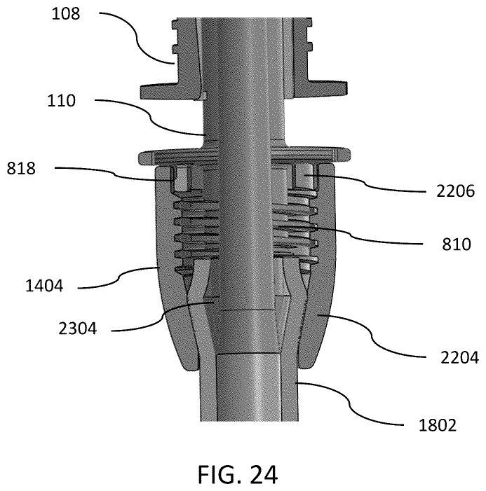

FIG. 24 is a cross-sectional side view of a tube retainer 1404 fully screwed onto a threaded tube barb 810 with a tube 1802 secured and sealed in place and with the anti-rotation features 2206 on the tube retainer 1404 engaged with a retainer locking tab 818 of the threaded tube barb 810. In this position, the smooth frustum portion 2204 of the threaded tube retainer 1404 compresses the tube 1802 against the barbed end 2304 of the valve connector 110 making the seal and keeping the connection secure around the circumference of the connection.

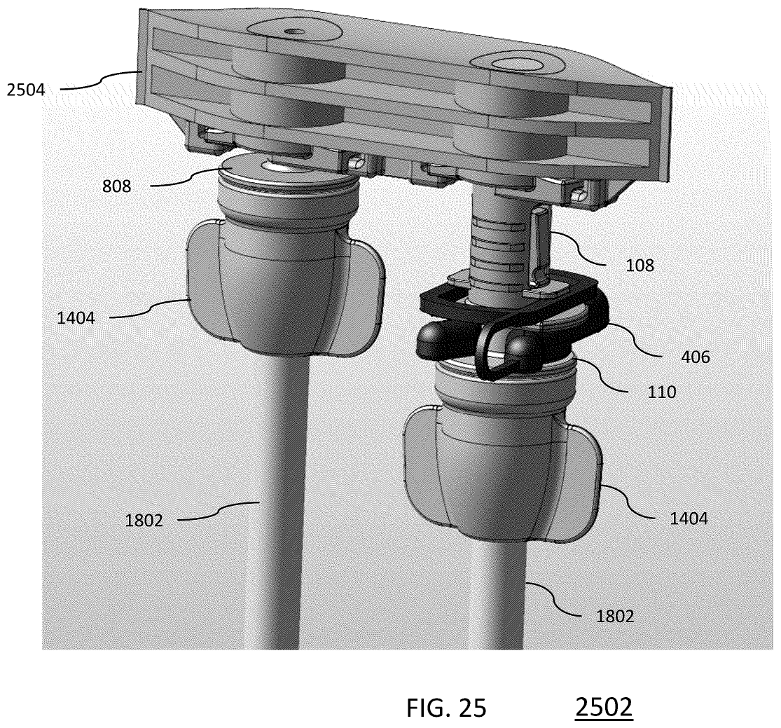

FIG. 25 is a perspective view of a canoe assembly 2502 having a two-port canoe 2504. The sub-assembly on the left has a threaded, fixed port fitting 808 mounted into the canoe 2504 and configured with a tube 1802 secured in place by a tube retainer 1404. The sub-assembly on the right has a port sizer 108 mounted into the canoe 2504 and configured with a valve connector 110 secured in the “closed” configuration by a tethered safety clip 406 and configured with a tube 1802 secured in place by a tube retainer 1404.

In certain embodiments, the bag chamber 112 may be made of multiple layers of HDPE/EVOH/Nylon or COC—PE/Cyclic Olefin for non-binding. The various other plastic elements may be made of polyethylene (PE), polypropylene (PP), polyvinylidene difluoride (PVDF), or other barrier and breathable layers.

### Alternative Ports and Port Fittings

Although port fittings and port sizers have been described in the context of so-called two-dimensional (2D) bags having canoes, such as bag assembly 100 of FIG. 1, in alternative embodiments, port fittings of the disclosure can be used in the context of other types of containers, such as (without limitation) so-called three-dimensional (3D) bags, bottles, and carboys.

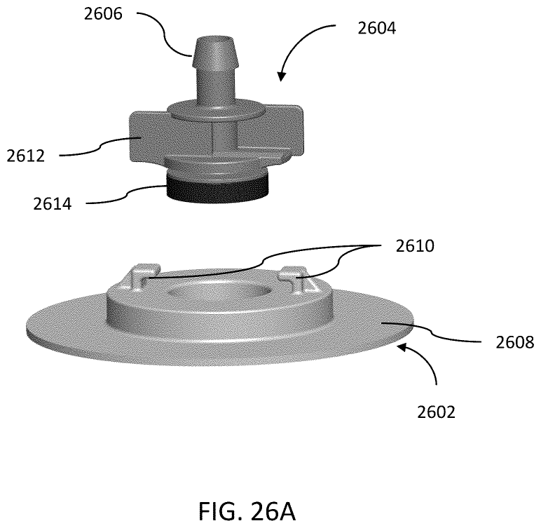

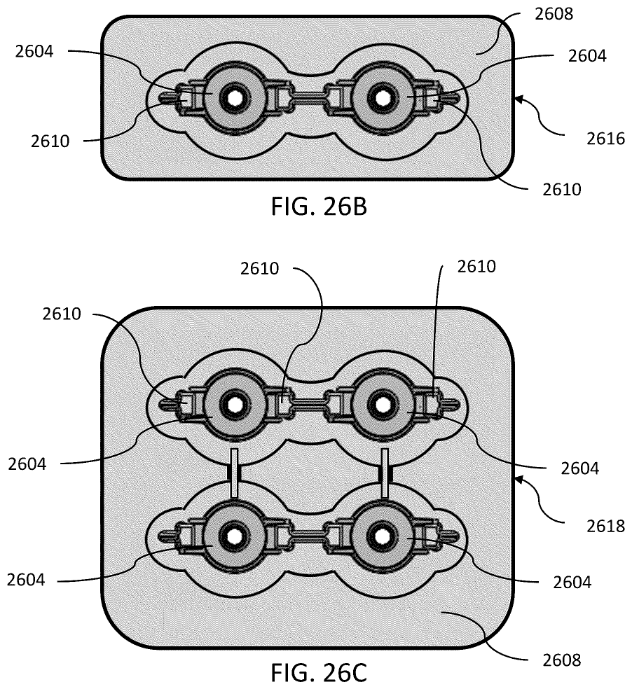

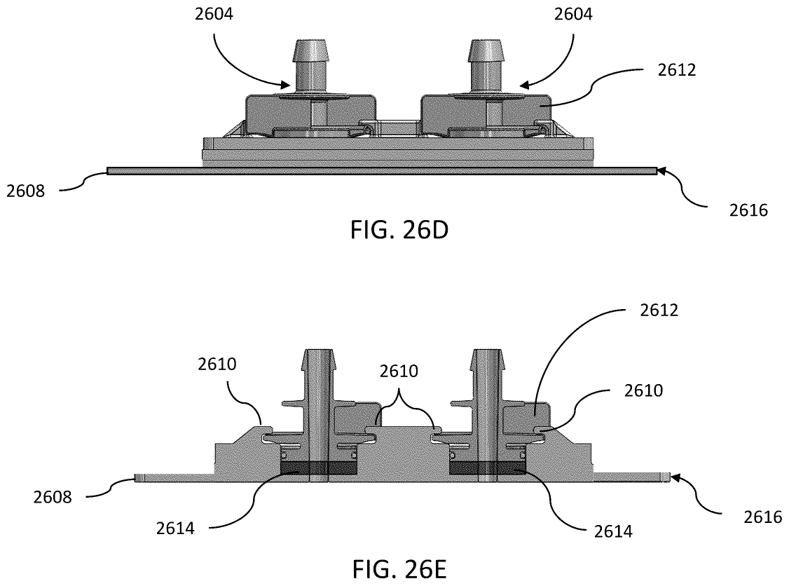

FIG. 26A is an exploded perspective view of a single-fitting port 2602 configured to receive a single, fixed port fitting 2604 having a tube barb 2606. The port 2602 has (i) a port welding flange 2608 at which the port 2602 is thermally welded, e.g., to a 3D bag or bottle/carboy cap (not shown) and (ii) retention clips 2610 that are analogous to the retention clips 506 of FIG. 5. The port 2602 may be made of any suitable material, such as (without limitation) thermoplastic elastomers, thermoplastic vulcanites, thermosets, and fluoropolymers. The port fitting 2604 has two opposing locking arms 2612 that engage the retention clips 506 to lock the port fitting 2604 onto the port 2602. The port fitting 2604 receives a septa gasket (i.e., a combined gasket/O-ring) 2614 that forms a seal between the port fitting 2604 and the port 2602.

FIG. 26B is a top-down view of a two-fitting port 2616 configured to receive two instances of the port fitting 2604, and FIG. 26C is a top-down view of a four-fitting port 2618 configured to receive four instances of the port fitting 2604.

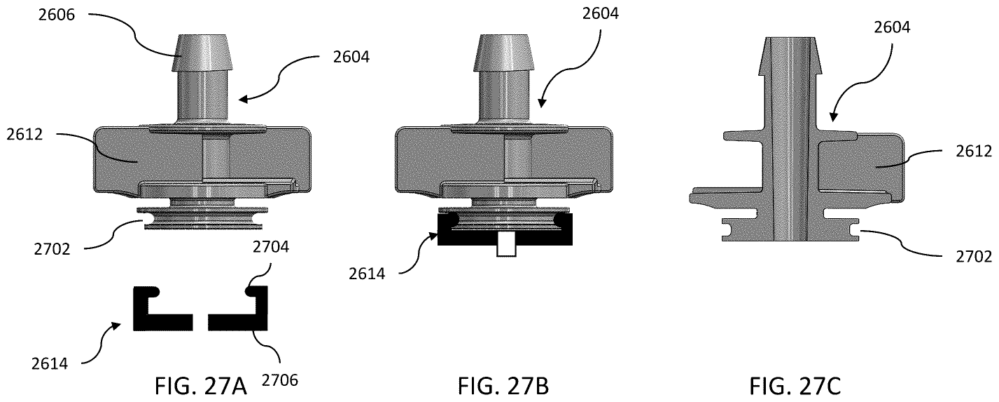

FIGS. 26D and 26E are side and cross-sectional (through the center line) views, respectively, of the two-fitting port 2616 with port fittings 2604 of FIG. 26B and septa gaskets 2614. FIGS. 27A and 27B are exploded and assembled views, respectively, showing a side view of the fixed port fitting 2604 and a cross-sectional view of a septa gasket 2614. FIG. 27C is a cross-sectional side view of the fixed port fitting 2604.

As shown in FIGS. 27A-27C, port fitting 2604 has an O-ring groove 2702, and septa gasket 2614 has an O-ring portion 2704 that is received by the O-ring groove 2702 and a gasket portion 2706 that abuts the bottom of the port fitting 2604. Note that septa gasket 2614 of FIG. 26E and septa gasket 2614 of FIGS. 27A and 27B provide the same function of forming seals to prevent leakage between the port fitting 2604 and the corresponding port X090.

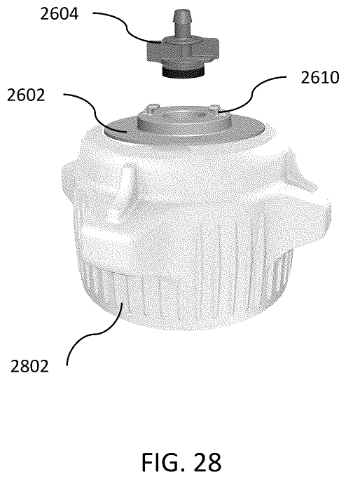

FIG. 28 is a partially exploded view of the port fitting 2604 and the single-fitting port 2602 mated with a cap 2802 for a bottle or carboy (not shown). According to one possible assembly technique, the single-fitting port 2602 is inserted into the bottom of the cap 2802 with the screwing down of the cap 2802 onto the bottle/carboy helping to form the seal between the single-fitting port 2602 and the cap 2802. Another possible assembly technique is to weld the single-fitting port 2602 to the cap 2802. Note that, in general, caps may be configured with single-or multi-fitting ports X090.

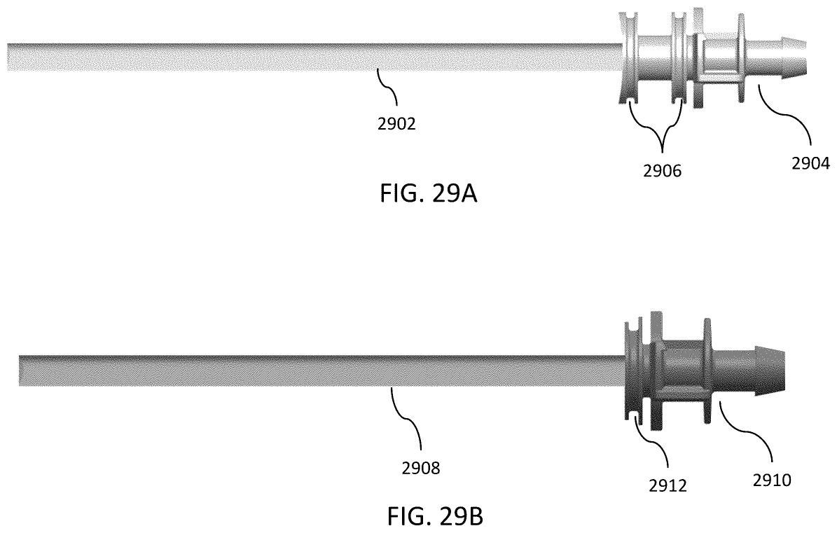

FIG. 29A is a side view of a dip tube 2902 having a dip tube fitting 2904 with two O-ring grooves 2906 designed to receive two O-rings before the assembly is inserted into a suitable port, and FIG. 29B is a side view of a dip tube 2908 having a dip tube fitting 2910 with a single septa gasket groove 2912 designed to receive the septa gasket 2614 of FIG. 26A before the assembly is inserted into a suitable port. The fittings 2904 and 2910 enable the dip tubes 2902 and 2908 to be mounted onto containers having suitable ports of the present disclosure, such as (without limitation) bags, bottles, and carboys.

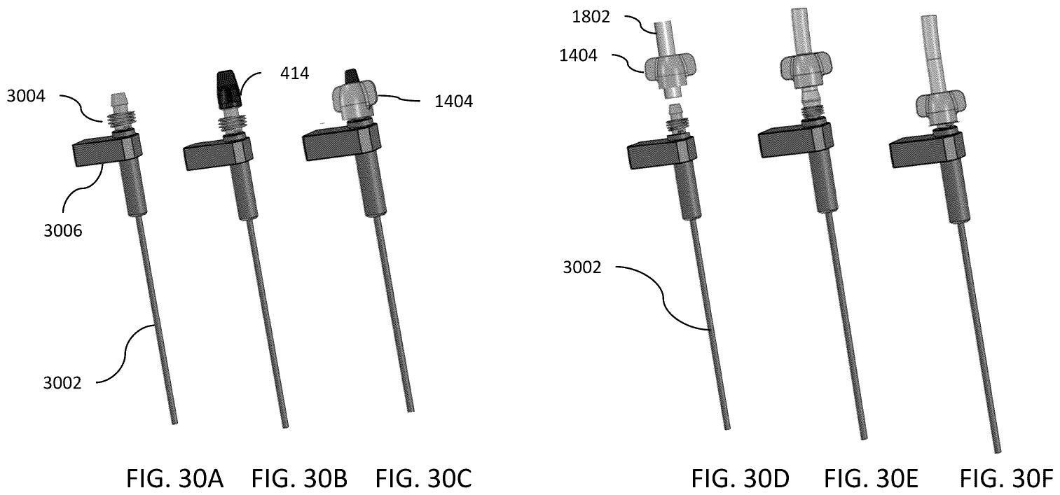

FIG. 30A is a perspective view of a filling needle 3002 having a threaded connector 3004 similar to the threaded tube barb 810 and a mounting bar 3006. FIG. 30B is a perspective view of the filling needle 3002 with a dust cap 414. FIG. 30C is a perspective view of the filling needle 3002 with both the dust cap 414 and a threaded tube retainer 1404. FIGS. 30D-30F are perspective views representing a tube 1802 being mounted onto the filling needle 3002 using the threaded tube retainer 1404.

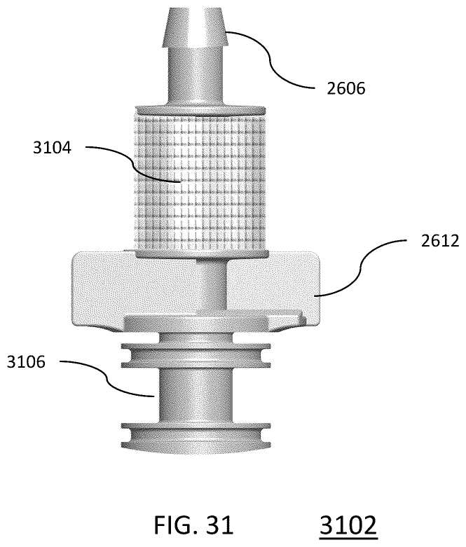

FIG. 31 is a side view of a vented port fitting 3102 having a filter housing 3104 that enables either one-way or two-way air flow into or out of the port fitting channel. The port fitting base 3106 is similar to analogous to the structure of the other port fittings of the disclosure. In one embodiment, the filter housing 3104 allows one-way air flow out of the port fitting channel, thereby enabling air to be vented from the container to which the port fitting 3102 is mounted as the container is being filled with liquid.

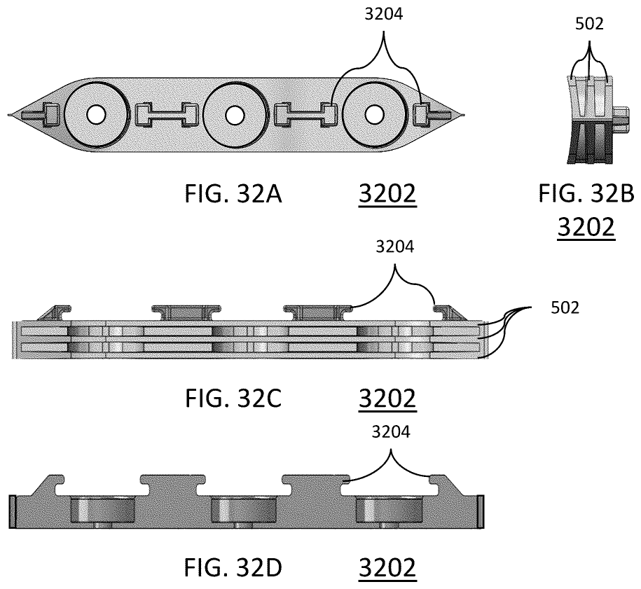

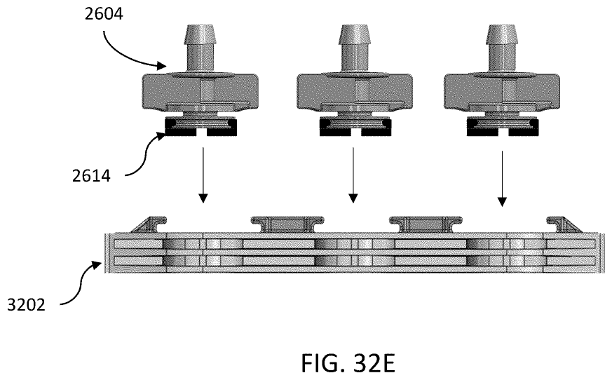

FIGS. 32A, 32B, 32C, and 32D show top-down, end, X-ray side, and cross-sectional side views of a three-port canoe 3202 according to an alternative embodiment. FIG. 32E is an exploded side view of the three-port canoe 3202 ready to receive three fixed port fittings 2604 having septa gaskets 2614 to form seals between the canoe 3202 and the port fittings 2604. With a reduced profile compared to canoe 114, canoe 3202 has only three weld bars 502. Note that the retention clips 3204 have a sturdier design than the retention clips 506 of the canoe 114.

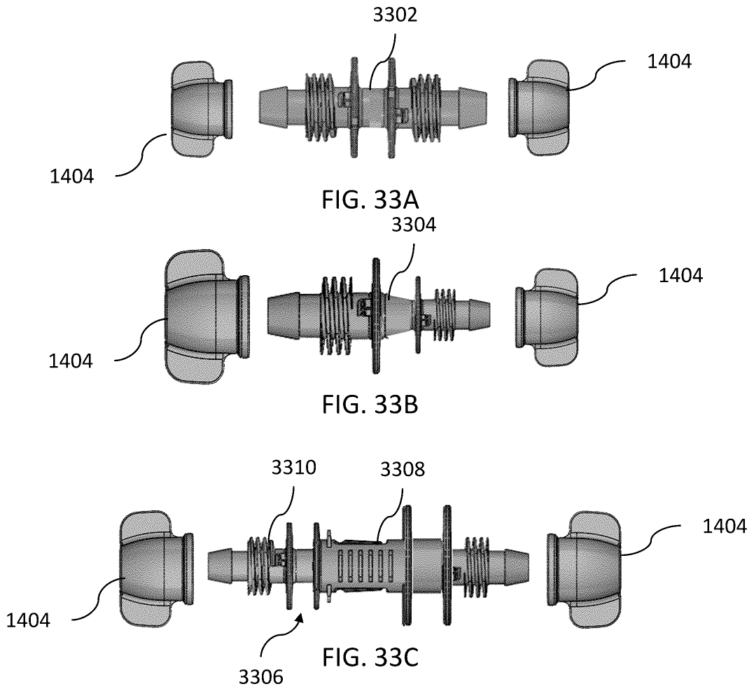

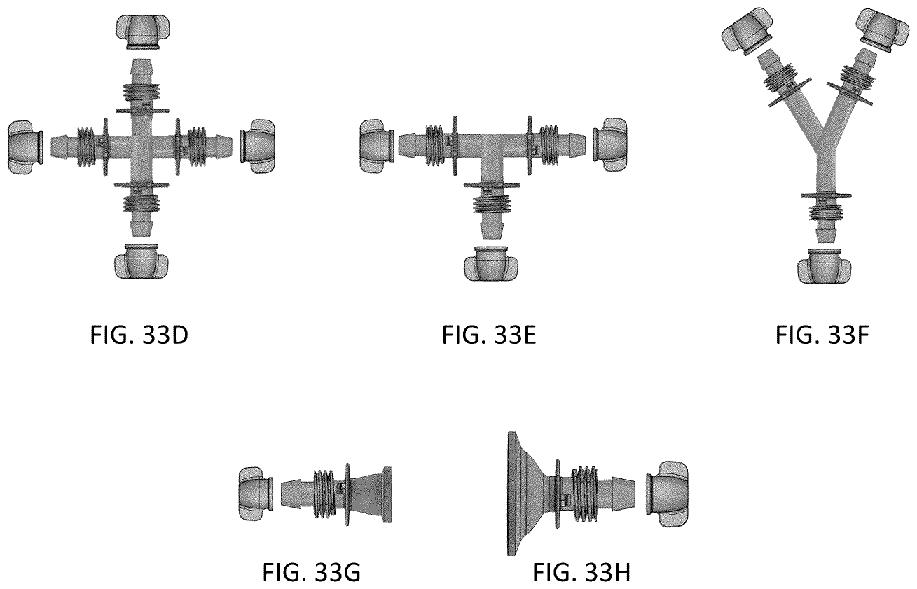

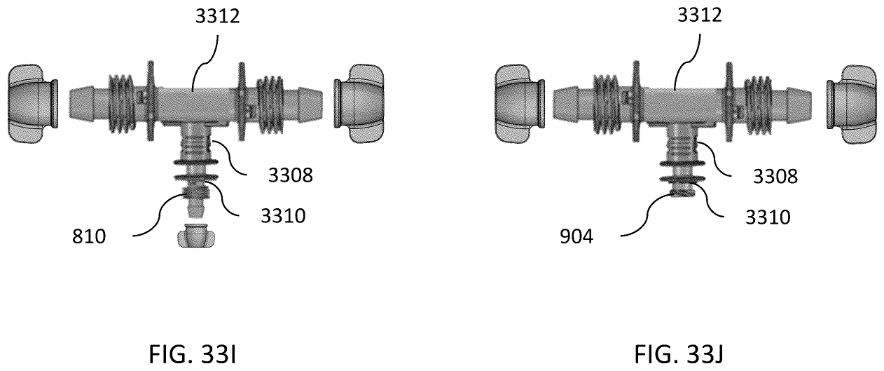

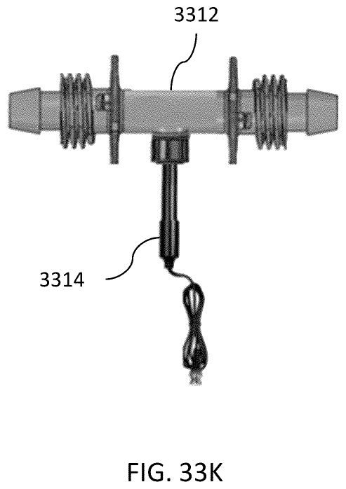

The tube retainer system of the present disclosure, which include a threaded tube barb 810 and a threaded tube retainer 1404, can also be applied in fixed tube-to-tube connectors, such as connector 3302 and reducer 3304 shown in the side view of FIGS. 33A and 33B, respectively. FIG. 33C is a side view of a two-piece tube-to-tube connector 3306 having a port sizer analog 3308 and a valve connector analog 3310 that provide flow-valve functionality. FIGS. 33D-33F show side views of retainer-ready cross, tee, and wye tube-to-tube connectors, respectively. FIGS. 33G and 33H show side views of retainer-ready tri-clamp tube barb connectors. FIGS. 33I and 33J show side views of two different three-piece tube-to-tube TEE connectors, each having a tube-to-tube connector analog 3312, a port sizer analog 3308, and a valve connector analog 3310 that provide flow-valve functionality at their lower ports with a threaded tube barb and a luer lock, respectively.

FIG. 33K is a side view of the three-port tube-to-tube connector analog 3312 of FIGS. 33I and 33J with a probe 3314 connected to the lower port to enable fluid path measurements.

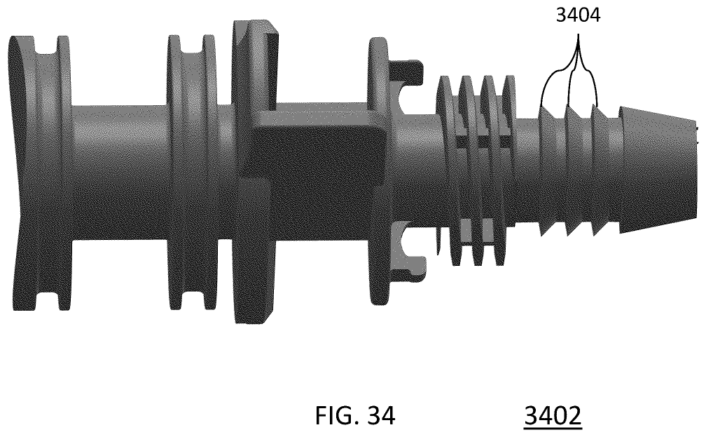

FIG. 34 is a side view of a fixed port fitting 3402 having a threaded tube barb with one or more (in this case, three) tube grippers 3404 that further improve the grip of the port fitting 3402 to flexible tubing (not shown).

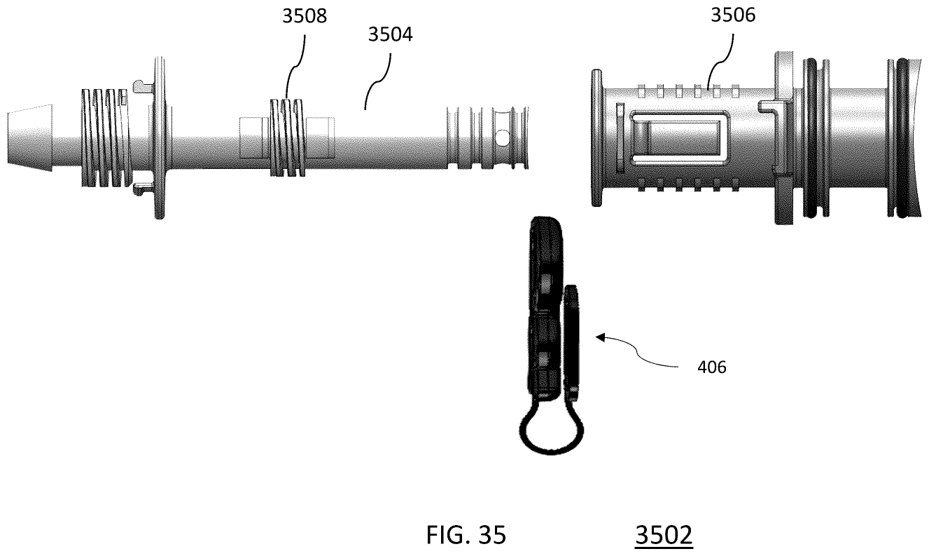

FIG. 35 is an exploded side view of a reconfigurable assembly 3502 comprising valve connector 3504, port sizer 3506, and tethered safety clip 406. Valve connector 3504 is similar to valve connector 110 except that valve connector 3504 has exterior threading 3508, and port sizer 3506 is similar to port sizer 108 except that port sizer 3506 has interior threading (not shown) that is designed to engage the threading 3508 of the valve connector 3504.

In this embodiment, the valve connector 3504 can be inserted into the port sizer 3506 and then rotated clockwise to a closed position in which the assembly 3502 is configured in a closed configuration that prevents fluid from flowing through the assembly 3502. The valve connector 3504 can then be further rotated clockwise to an open position in which the assembly 3502 is configured in an open configuration that enables fluid to flow through the assembly 3502. The valve connector 3504 can then be rotated counterclockwise to return the assembly 3502 to the closed configuration. In addition, valve connector 3504 can be further rotated counterclockwise to completely remove the valve connector 3504 from the port sizer 3506. When assembled, the tethered safety clip 406 can be used in a manner analogous to that described previously to prevent the valve connector 3504 from inadvertently advancing from the closed position to the open position.

Note that, in order to enable the valve connector 3504 to rotate inside the port sizer 3506, certain features, such as the locking tabs 706/708 and the keyway guide 718 of the valve connector 110 and the keyway 616 and the locking clip 606 of the port sizer 108, may need to be modified or even removed in the designs of the valve connector 3504 and the port sizer 3506.

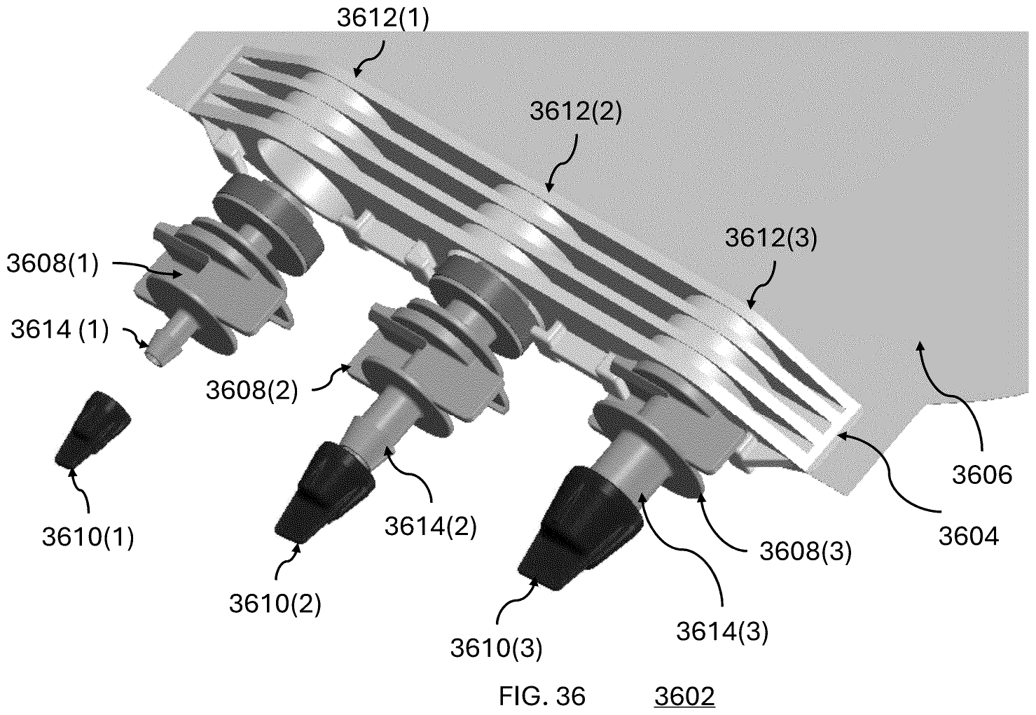

FIG. 36 is a perspective view of the bottom of container (in this case, a bag assembly) 3602 according to an alternative embodiment. FIG. 36 shows a three-port connector (in this case, a canoe) 3604 welded to the bottom of a bag 3606 and three differently sized port fittings 3608(1)-3608(3) with three corresponding dust caps 3610(1)-3610(3). In particular, FIG. 36 shows the small-sized port fitting 3608(1) before being inserted into the first port 3612(1) of the canoe 3604 with the dust cap 3610(1) before being applied onto the corresponding tube barb 3614(1), the medium-sized port fitting 3608(2) being inserted into the second port 3612(2) of the canoe 3604 with the dust cap 3610(2) being applied onto the corresponding tube barb 3614(2), and the large-sized port fitting 3608(3) after being fulling mounted within the third port 3612(3) of the canoe 3604 with the dust cap 3610(3) fully applied onto the corresponding tube barb 3614(3). Note that, as described further below, a port fitting 3608 is locked onto the canoe 3604 by inserting the port fitting into a canoe port 3612 and then rotating the port fitting 3608 one quarter turn with respect to the canoe 3604 until an audible click is heard indicating that the port fitting 3608 is locked into place.

Note that, although the three port fittings 3608 have different size tube barbs 3614 for receiving corresponding different size tubing (not shown in FIG. 36), the three ports 3612 of the canoe 3604 all have the same size and each port fitting 3608 is able to be mounted into any of the three canoe ports 3612. The three port fittings 3608 are fixed port fittings that provide a fixed opening into the bag assembly 3602.

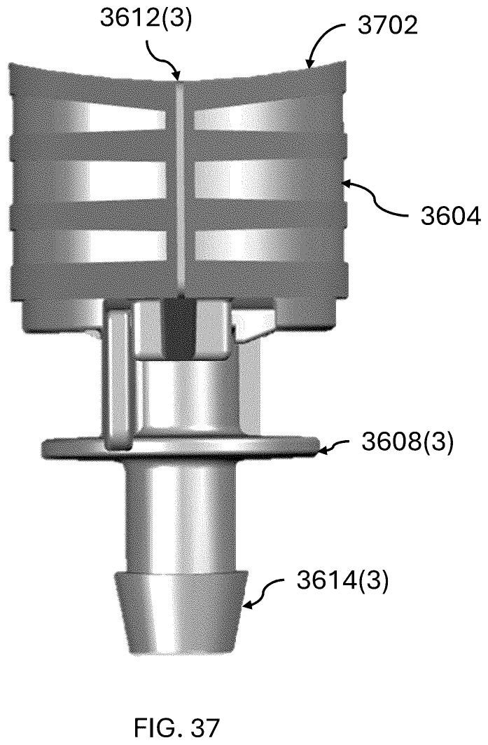

FIG. 37 is an end view of the port fitting 3608(3) of FIG. 36 fully mounted into the third port 3612(3) of the canoe 3604. As shown in FIG. 37, the top 3702 of the canoe 3604 has curvature that assists in (i) avoiding dead-pooling of liquid at the bottom of the bag 3606 and (ii) draining liquid from the bag assembly 3602.

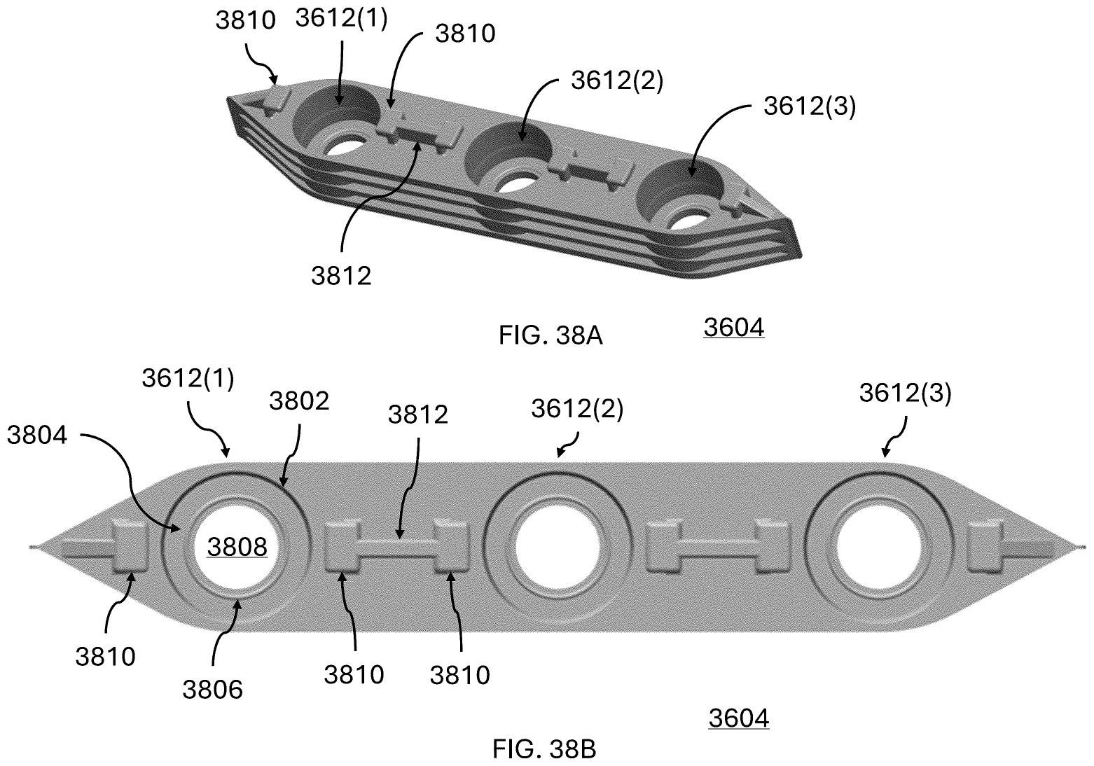

FIGS. 38A and 38B are a perspective view and a plan view, respectively, of the three-port canoe 3604 of FIG. 36. As shown in FIG. 38B, each port 3612 in the canoe 3604 has an outer rim 3802 that defines a recessed, circular, inner seat 3804 having a raised, circular septa-sealing ring 3806 and a circular through-hole 3808. Each port 3612 also has two opposing retention clips 3810. Note that the adjacent retention clips 3810 for adjacent ports 3612 have connecting structure 3812 that improves the strength and durability of the retention clips 3810.

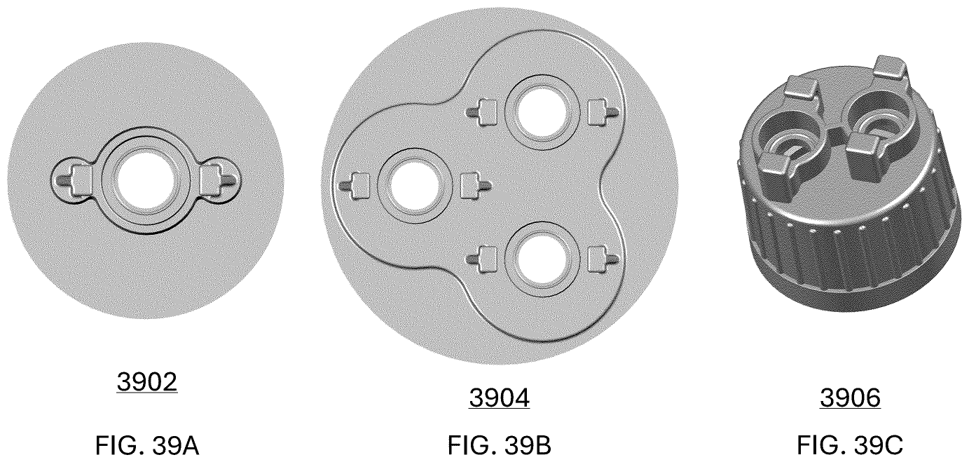

FIG. 39A is a plan view of a one-port 3D bag connector 3902, FIG. 39B is a plan view of a three-port 3D bag connector 3904, and FIG. 39C is a perspective view of a two-port bottle cap connector 3906, where each of the ports of those connectors has similar features as the ports 3612 of FIG. 38B that enable any of the port fitting 3612 of FIG. 36 to be configured to any port of those connectors.

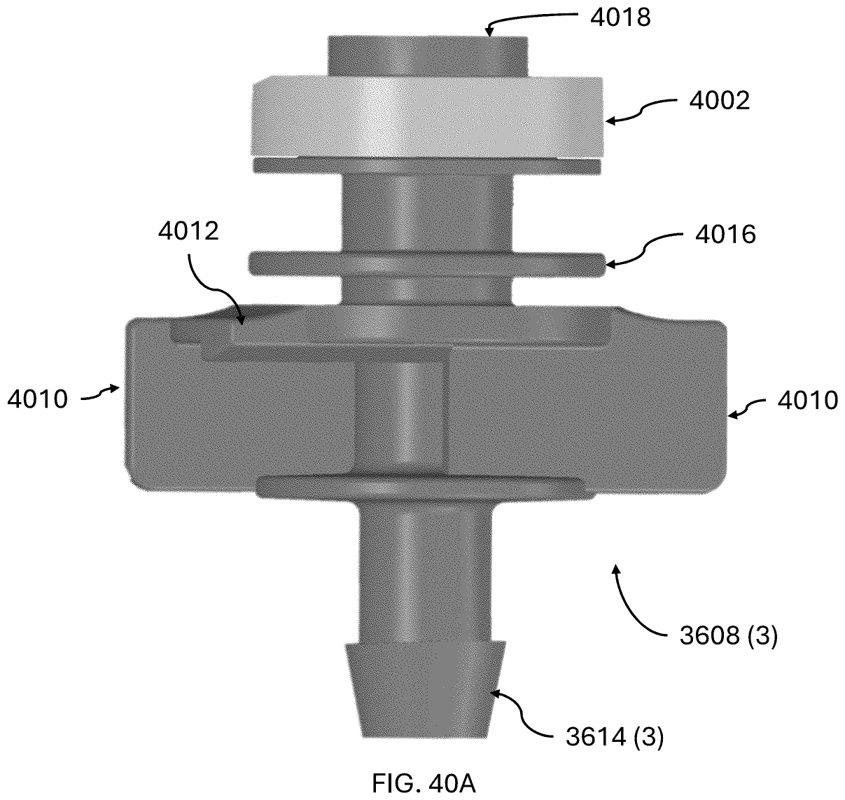

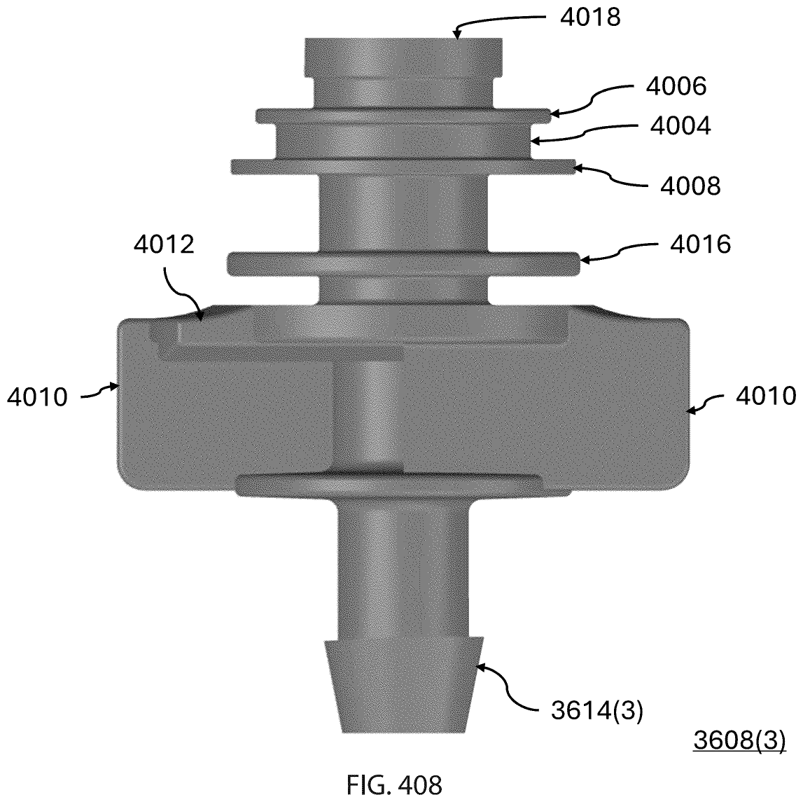

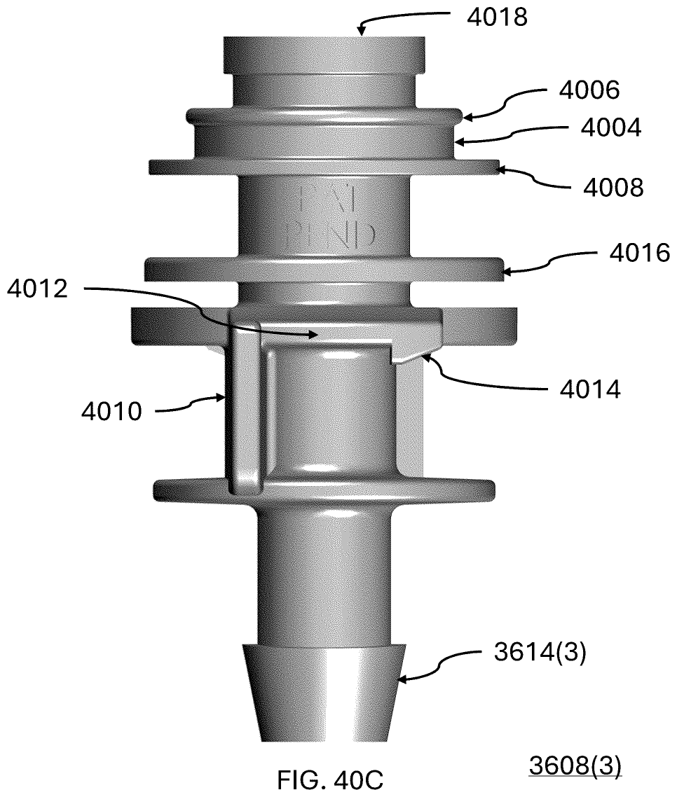

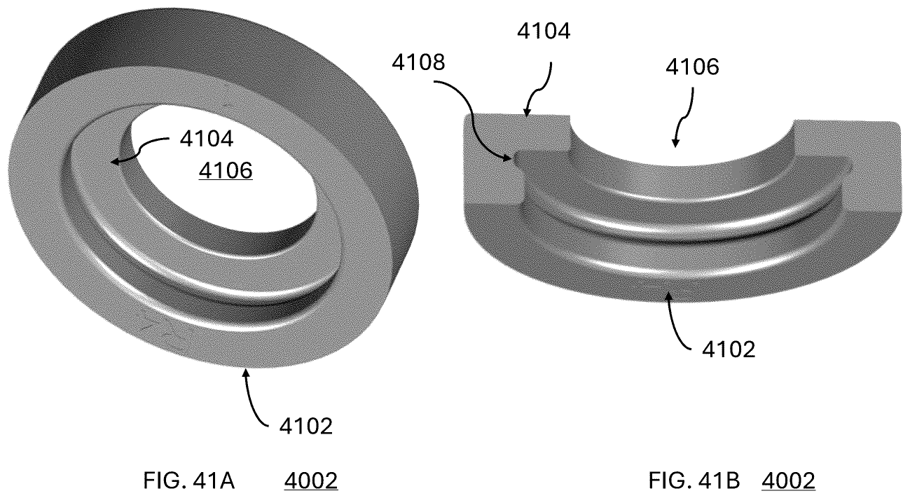

FIG. 40A is a side view of the port fitting 3608(3) of FIG. 36 configured with a unitary (i.e., one-piece) septa gasket 4002; FIG. 40B is a side view of the port fitting 3608(3) of FIG. 40A without the septa gasket 4002; FIG. 40C is a side view of the port fitting 3608(3) of FIG. 40B rotated 90 degrees about the vertical axis; FIG. 41A is a perspective view of the septa gasket 4002 of FIG. 40A; and FIG. 41B is a cutaway, perspective view of the septa gasket 4002 of FIG. 40A. The purpose of the septa gasket is to create a leak-proof seal between the port fitting 3608 and the canoe port 3612 to prevent liquid inside the bag assembly 3602 from inadvertently leaking out through the canoe port 3612 and the port fitting 3608.

As shown in FIGS. 41A and 41B, the septa gasket 4002 has a circular, O-ring portion 4102 connected to a circular, disk-shaped gasket portion 4104 having a circular through-hole 4106 and forming a circular recess 4108. The septa gasket 4002 is made of a suitable, resilient (e.g., elastomeric) material.

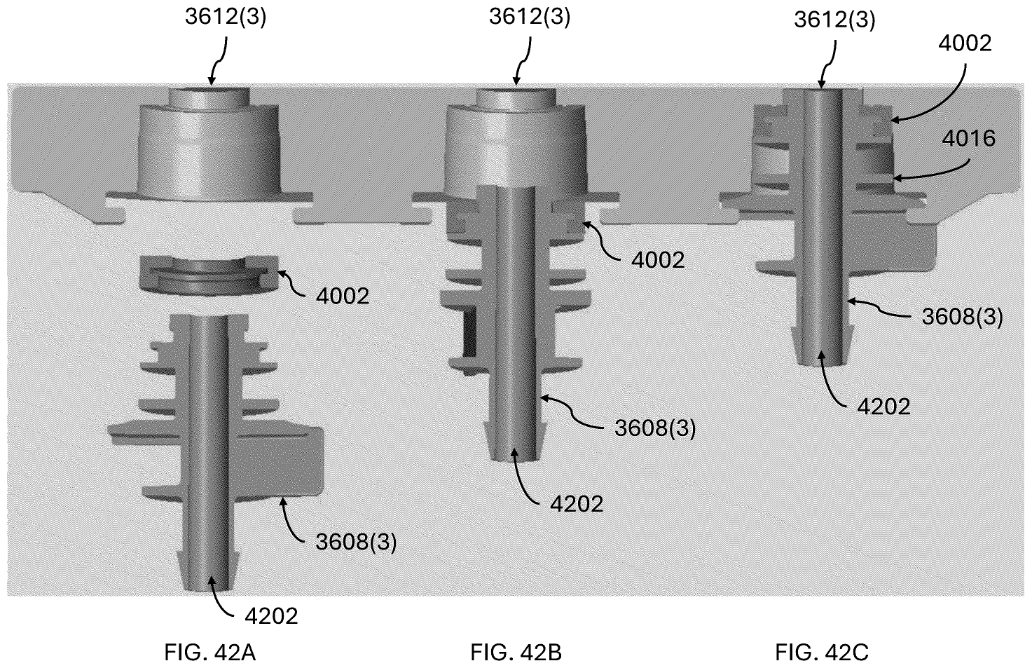

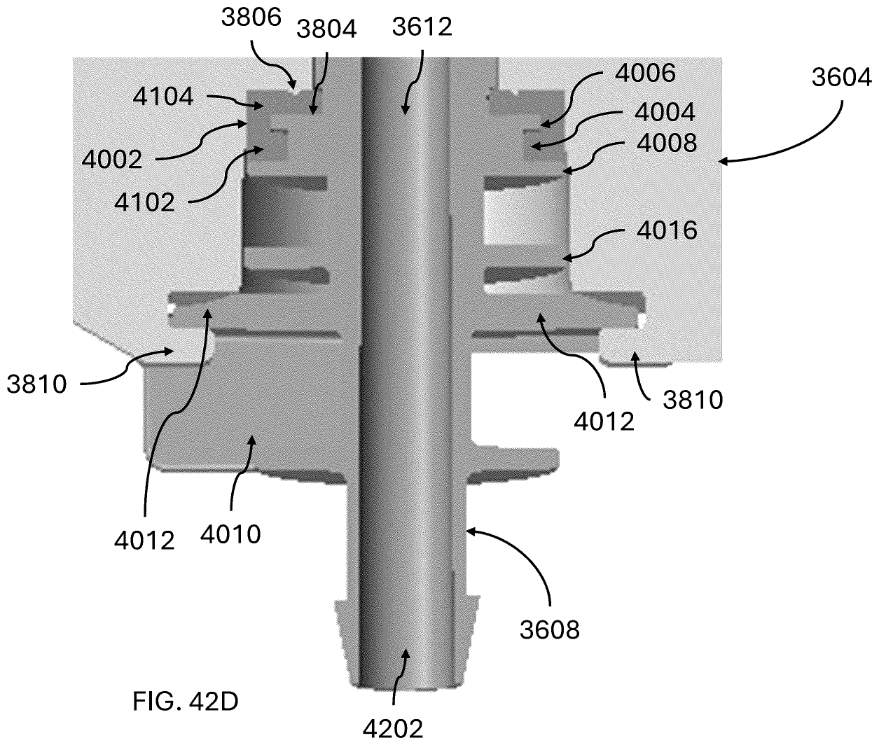

As shown in FIGS. 40B and 40C, the port fitting 3608(3) has a septa gasket groove 4004 defined between an upper rim 4006 and a middle rim 4008, where the septa gasket groove 4004 is designed to receive the O-ring portion 4102 of the septa gasket 4002 of FIG. 40A with the septa gasket's recess 4108 receiving the outer edge of the upper rim 4006 and the gasket portion 4104 positioned on top of the upper rim 4006, as shown in FIG. 42D. FIG. 40A shows the septa gasket 4002 configured onto the port fitting 3608(3).

In addition, as shown in FIGS. 40A-40C, the port fitting 3608(3) has two opposing wings 4010, each with a locking arm 4012 designed to engage one of the canoe's retention clips 3810 when the port fitting 3608(3) is fully mounted within the corresponding canoe port 3612(3) by inserting and then rotating the port fitting 3608(3) relative to the canoe port 3612(3). As shown in FIG. 40C, each locking arm 4012 has a detent 4014 that engages with the side of the retention clip 3810 to assist in retaining the port fitting 3608(3) within the canoe port 3612(3). In some embodiments, the locking arms 4012 are designed to prevent the port fitting 3608(3) from being removed from the canoe port 3612 without permanently damaging and rendering unusable either the port fitting or the canoe or both. In other embodiments, the locking arms 4012 are designed to prevent inadvertent removal, but still enable the port fitting 3608(3) to be removed purposely from the canoe port 3612.

The port fitting 3608(3) also has a lower rim 4016 that, as shown in FIG. 42C, fits within the canoe port 3612(3) when the port fitting is mounted within the canoe port. During normal use, the mounted fitting 3608(3) will be subject to side forces from connected tubing with sometimes relatively heavy components connected to the tubing. The lower rim 4016 provides stability for the mounted fitting 3608(3) with respect to those side forces.

Although hard to see in FIG. 40C, the top 4018 of the port fitting 3608(3) has curvature that matches the curvature at the top BA of the canoe 3604 to assist in (i) avoiding the dead-pooling of liquid at the bottom of the bag 3606 and (ii) draining liquid from the bag assembly 3602.

FIG. 42A is an exploded, cut-away view of the port fitting 3608(3) of FIG. 36, the septa gasket 4002, and the canoe port 3612(3); FIG. 42B is a cut-away view of the port fitting 3608(3) configured with the septa gasket 4002 and partially inserted into the canoe port 3612(3); and FIG. 42C is a cut-away view of the port fitting 3608(3) configured with the septa gasket 4002 and fully mounted within the canoe port 3612(3). FIGS. 42A-42C all show the cylindrical passage 4202 through port fitting 3608(3) that enables liquid to flow out of the bag 3606 of FIG. 36 via the port fitting 3608(3).

FIG. 42D shows a zoomed-in, cross-sectional view of a port fitting 3608 fully mounted within a canoe port 3612 of the canoe 3604 of FIG. 36. As shown in FIG. 42D, the port fitting's locking arms 4012 are engaged with the canoe port's retention clips 3810. The septa gasket 4002 fills the port fitting's septa gasket groove 4004, wraps around the port fitting's upper rim 4006, and covers the upper surface of the upper rim 4006. As the port fitting 3608 is inserted into and then rotated relative to the canoe port 3612, the septa gasket 4002 is squeezed between the structure of the port fitting 3608 and the structure of the canoe port 3612, resulting in complementary outward forces exerted by the resilient septa gasket 4002 onto those structures to create the septa gasket's leak-proof seal between the port fitting 3608 and the canoe port 3612. That seal is further improved by the septa-sealing ring CE of the canoe port's seat CD forming a corresponding septa-seal depression in the resilient material of the gasket portion 4104 of the septa gasket 4002.

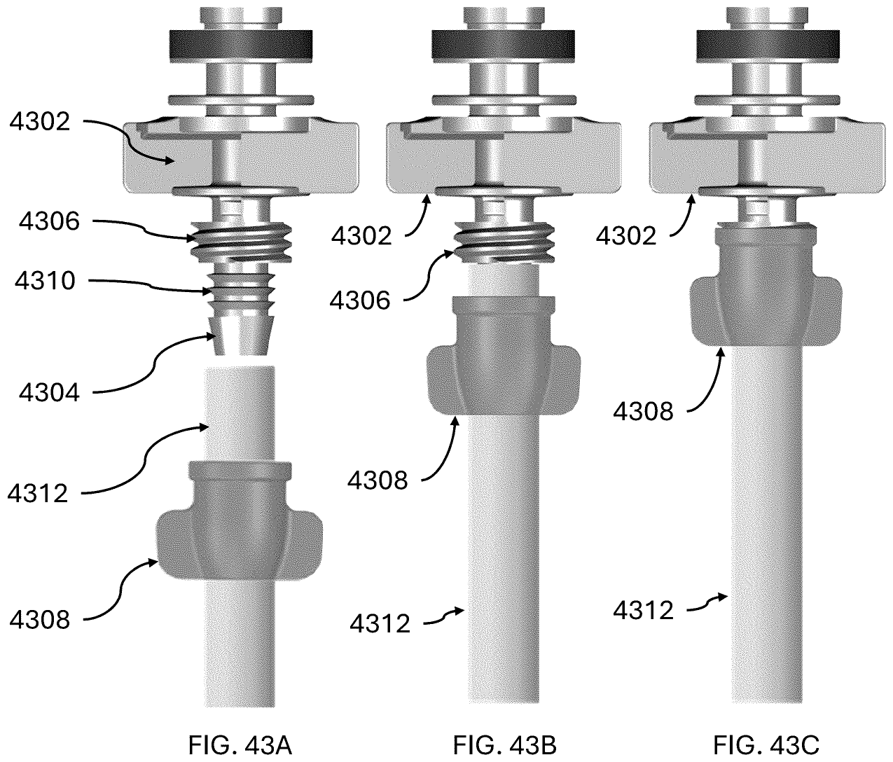

FIGS. 43A-43C are side views of an alternative, fixed port fitting 4302 having a threaded tube barb 4304 with (i) external threading 4306 for engaging with the internal threading (not shown) of a tube retainer 4308 and (ii) three tube grippers 4310 to retain a flexible tube 4312 and assist in forming a leak-proof seal between the port fitting 4302 and the tube 4312. Alternative embodiments may have a different number of tube grippers 4310. In particular, FIG. 43A shows the tube 4312 inserted through the tube retainer 4308; FIG. 43B shows the tube 4312 rammed over the end of the tube barb 4304 before the tube retainer 4308 is rotated onto the threading 4306; and FIG. 43C shows the full assembled unit after the tube retainer 4308 has been rotated onto the threading 4306.

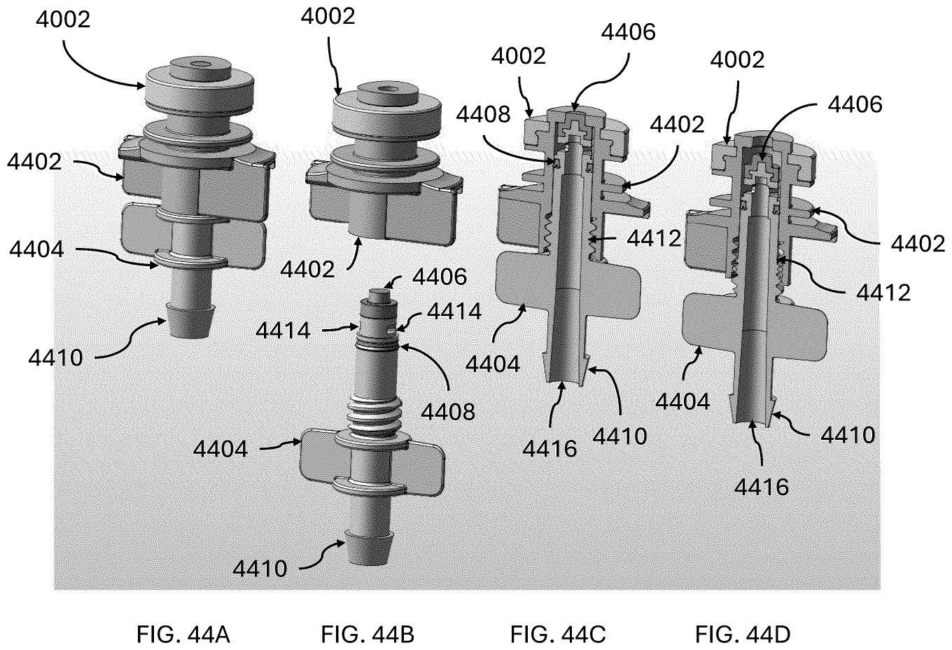

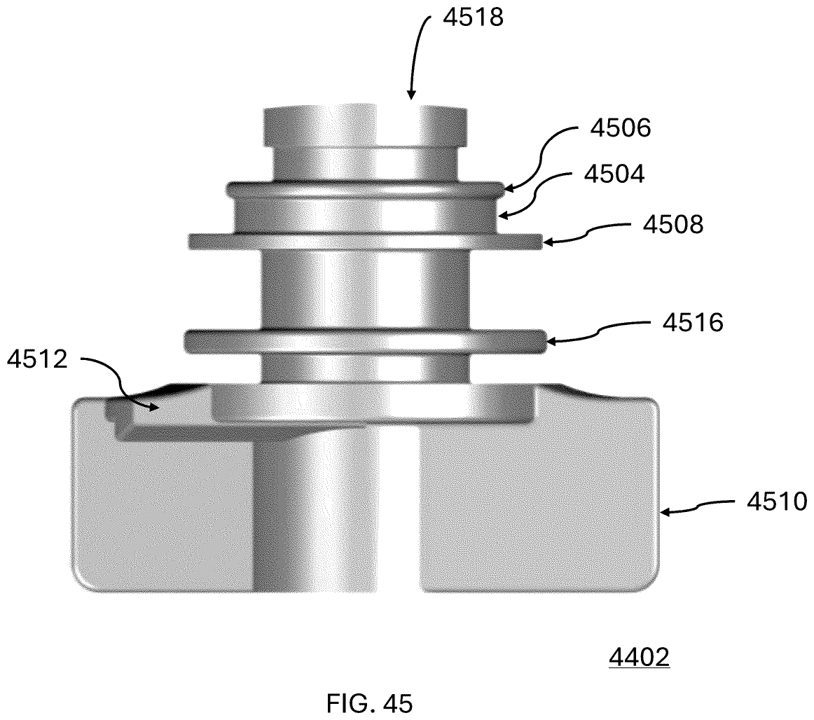

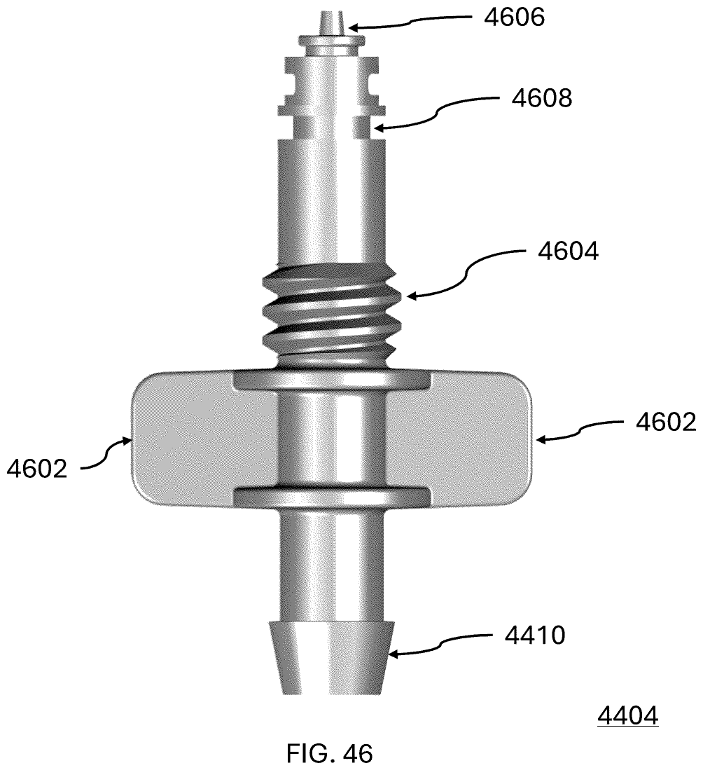

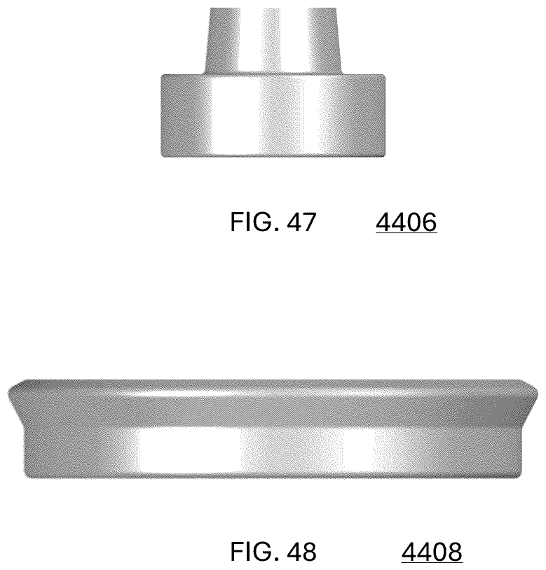

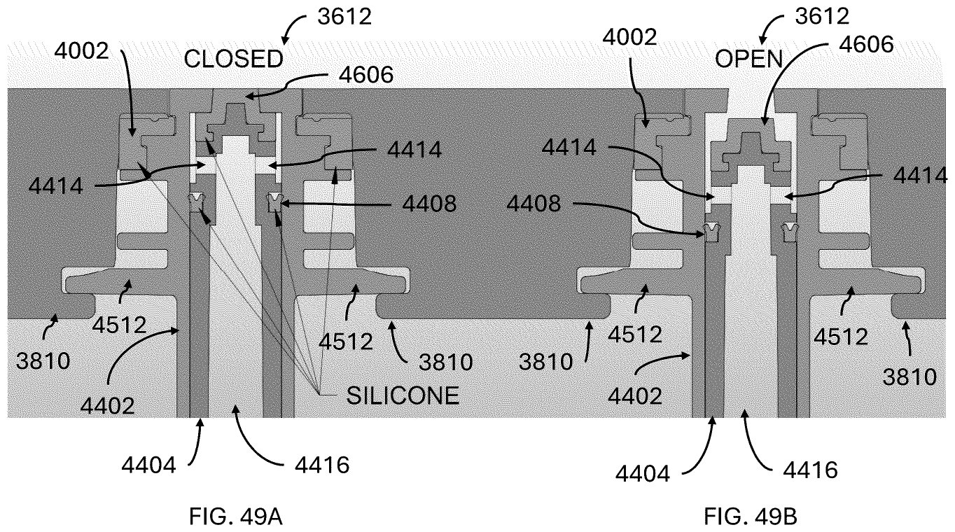

FIG. 44A is a perspective view of an assembly that includes an alternative port sizer 4402 and a valve connector 4404. FIG. 44B is a perspective view of the port sizer 4402 and the valve connector 4404. FIG. 44C is a cutaway, perspective view of the assembly of FIG. 44A in its fully closed configuration, and FIG. 44D is a cutaway, perspective view of the assembly of FIG. 44A in an opened configuration. FIG. 45 is a side view of the port sizer 4402, FIG. 46 is a side view of the valve connector 4404, FIG. 47 is a side view of the valve connector's (e.g., silicone) sealing gasket 4406, and FIG. 48 is a side view of the valve connector's (e.g., silicone) wiper ring 4408. FIG. 49A is a cross-sectional side view of the assembly of FIG. 44C mounted within a canoe port 3612 and in the closed configuration, and FIG. 49B is a cross-sectional side view of the assembly of FIG. 44D mounted within a canoe port 3612 and in the opened configuration.

As shown in FIG. 45, the upper end of the port sizer 4402 is similar to the upper end of the port fitting 3608(3) shown in FIG. 40C with analogous features having analogous labels. As such, the port sizer 4402 can be configured with the same septa gasket 4002 in the same manner and with the same function as with the port fitting 3608(3). In addition, the port sizer 4402 has wings 4510 and locking arms 4512 that are similar to the corresponding elements of the port fitting 3608(3) with the same function of engaging with the retention clips 3810 of a canoe port 3612, as represented in FIGS. 49A and 49B.

Unlike the port fitting 3608(3), however, the port sizer 4402 does not have a tube barb. Instead, port sizer 4402 receives the valve connector 4404, which does have a tube barb 4410, as shown in FIG. 46. The valve connector 4404 has opposing wings 4602 and external threading 4604 that is designed to engage with internal threading 4412 of the port sizer 4402, as shown in FIGS. 44C and 44D. The top 4606 of the valve connector 4404 is designed to receive the sealing gasket 4406. The valve connector 4404 also has a wiper-ring groove 4608 designed to receive the wiper ring 4408 (or, alternatively, an O-ring). Between the top 4606 and the wiper-ring groove 4608, the valve connector 4404 has one or more (e.g., two) openings 4414 for fluid flow, as shown in FIGS. 44B, 49A, and 49B.

With the valve connector 4404 configured with the sealing gasket 4406 and the wiper ring 4408, as shown in FIG. 44B, the valve connector 4404 is inserted into and then rotated with respect to the port sizer 4402 to engage the valve connector's external threading 4604 with the port sizer's internal threading 4412 with the wiper ring 4408 forming a leak-proof seal between the valve connector 4404 and the port sizer 4402.

As shown in FIGS. 44C and 49A, when the valve connector 4404 is fully engaged with the port sizer 4402 at a so-called closed position, the sealing gasket 4406 forms a leak-proof seal between the valve connector 4404 and the port sizer 4402 that prevents liquid from inadvertently flowing out of the bag assembly. Although not shown in the figures, this embodiment would include a mechanism for retaining the assembly in the closed configuration. One option could be similar to the tethered safety clip 406 of FIGS. 4C-4E.

As shown in FIGS. 44D and 49B, when the valve connector 4404 is only partially engaged with the port sizer 4402 at a so-called opened position, liquid in the bag assembly is able to flow through the opening in the canoe port 3612 around the sealing gasket 4406 and the top 4606 of the valve connector 4404 and then through the valve connector's openings 4414 and into the valve connector's passageway 4416. Note that, in this embodiment, the opened configuration is achieved by de-rotating the valve connector 4404 with respect to the port sizer 4402 to retract the valve connector 4404 within the port sizer 4402.

After being opened by de-rotating the valve connector 4404 to achieve the opened configuration shown in FIGS. 44D and 49B, the assembly can be re-closed by re-rotating the valve connector 4404 to re-achieve the closed configuration shown in FIGS. 44C and 49A. In this way, the flow of liquid out of the bag assembly can be successfully regulated. Significantly, the bag assembly 3602 is sealed off at the top of the canoe 3604 by the valve connector 4404 and the sealing gasket 4406, thereby avoiding the well-known dead-leg problem associated with conventional technology where liquid is undesirably trapped within the tubing between the bag and whatever conventional mechanism is used to close the tubing. The bag assembly 3602 of FIG. 36 is said to achieve zero dead leg, i.e., the elimination of retained liquid beyond the bag chamber. Any residual liquid within the valve connector's openings 4414 and passageway 4416 and the attached tubing (not shown) will drain away due to gravity

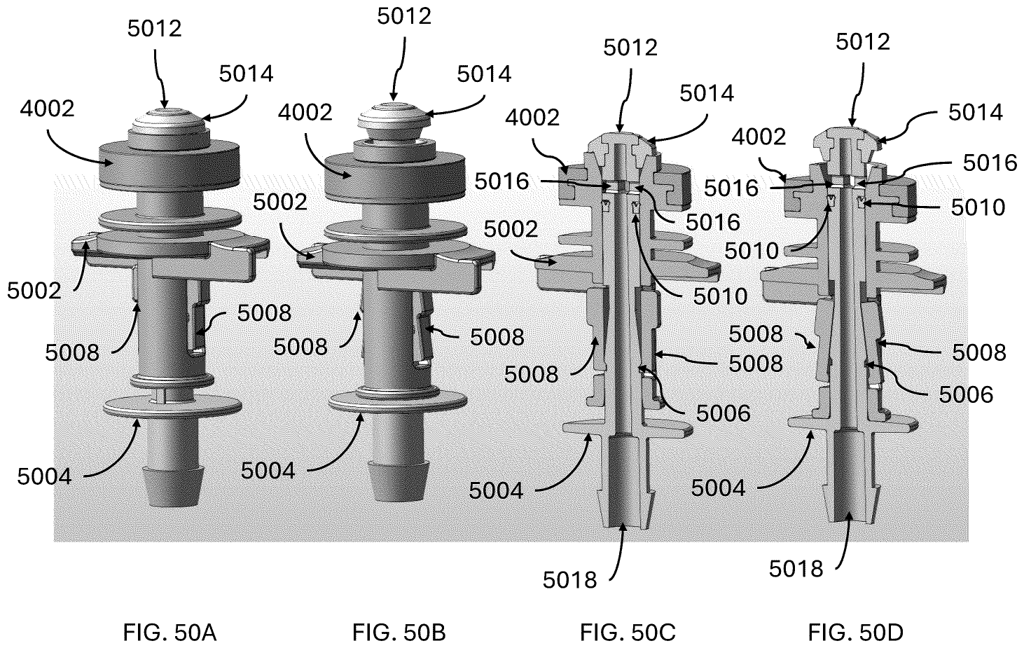

FIG. 50A is a perspective view of another assembly that includes another alternative port sizer 5002 and an alternative valve connector 5004, where the assembly is in its fully closed configuration. FIG. 50B is a perspective view of the assembly of FIG. 50A, but in its fully opened configuration. FIG. 50C is a cutaway, perspective view of the closed assembly of FIG. 50A, and FIG. 50D is a cutaway, perspective view of the opened assembly of FIG. 50B.

The upper end of the port sizer 5002 is similar to the upper ends of the port fitting 3608(3) and port sizer 4402 shown in FIGS. 40A-40C and 45. As such, the port sizer 5002 can be configured with the same septa gasket 4002 in the same manner and with the same function as with the port fitting 3608(3) and port sizer 4402. In addition, the port sizer 5002 has wings and locking arms that are similar to the corresponding elements of the port fitting 3608(3) and port sizer 4402 with the same function of engaging with the retention clips 3810 of a canoe port 3612.

The valve connector 5004 has a locking tab 5006 that engages with a pair of opposing locking clips 5008 of the port sizer 5002 to perform similar functions to those shown in the embodiments of FIGS. 6A-6C and 7A-7B to achieve the fully closed configuration of FIGS. 50A and 50C and the fully opened configuration of FIGS. 50B and 50D. Note that, in this embodiment, the opened configuration is achieved by extending the valve connector 5004 further within the port sizer 5002 as in the embodiment of FIGS. 6A-6C and 7A-7B.

As in the embodiment of FIGS. 44A-49B, the valve connector 5004 receives a wiper ring 5010 (or, alternatively, an O-ring) that forms a leak-proof seal between the valve connector 5004 and the port sizer 5002. As in the embodiment of FIGS. 44A-49B, the top 5012 of the valve connector 5004 receives a sealing gasket 5014 that forms a leak-proof seal between the valve connector 5004 and the port sizer 5002 when the assembly is in its closed configuration, as shown in FIGS. 50A and 50C, although the sealing gasket 5014 has a different shape from that of the sealing gasket 4406 of FIGS. 44A-49B.

As shown in FIGS. 50A and 50C, when the valve connector 5004 is inserted into the port sizer 5002 up to a closed position, the sealing gasket 5014 forms a leak-proof seal between the valve connector 5004 and the port sizer 5002 that prevents liquid from inadvertently flowing out of the bag assembly. As shown in FIGS. 50B and 50D, when the valve connector 5004 is inserted further into the port sizer 5002 up to its fully opened configuration, liquid in the bag assembly is able to flow through the through-hole in the canoe port (not shown) around the sealing gasket 5014 and the top 5012 of the valve connector 4404 and then through the valve connector's openings 5016 and into the valve connector's passageway 5018. Here, too, although not shown in the figures, this embodiment would include a mechanism for retaining the assembly in the closed configuration. One option could be similar to the tethered safety clip 406 of FIGS. 4C-4E.

After being opened by further inserting the valve connector 5004 within the port sizer 5002 to achieve the opened configuration shown in FIGS. 50B and 50D, the assembly can be re-closed by partially retracting the valve connector 5004 relative to the port sizer 5002 to re-achieve the closed configuration shown in FIGS. 50A and 50C. In this way, the flow of liquid out of the bag assembly can again be successfully regulated. Here, too, the valve connector 5004 and the sealing gasket 5014 close off the bag at the top of the canoe, again achieving zero dead leg and avoiding the dead-leg problem.

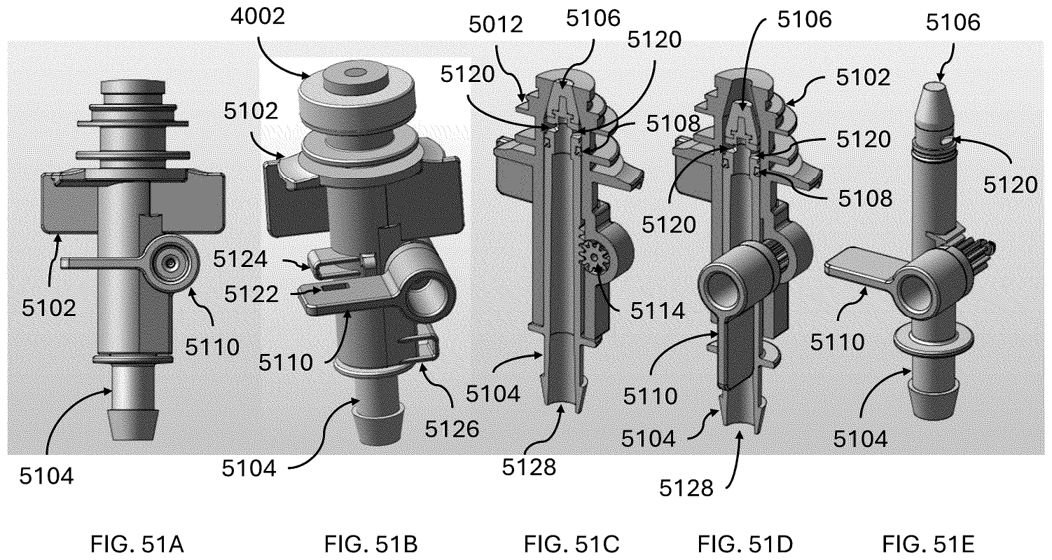

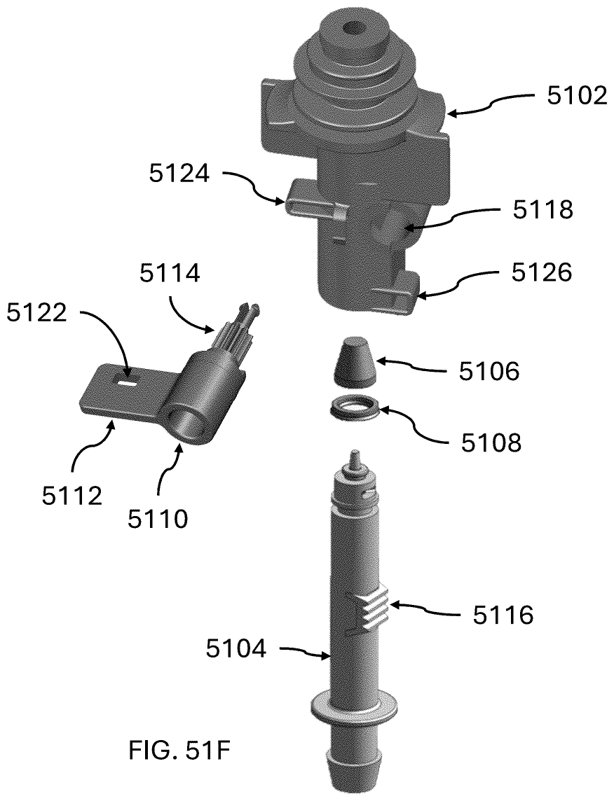

FIG. 51A is a side view of yet another assembly that includes yet another alternative port sizer 5102 and another alternative valve connector 5104, where the assembly is in its fully closed configuration. FIG. 51B is a perspective view of the assembly of FIG. 51A in its fully closed configuration. FIG. 51C is a cutaway, perspective view of the closed assembly of FIGS. 51A and 51B, and FIG. 51D is a cutaway, perspective view of the assembly of FIGS. 51A-51C, but in an opened configuration. FIG. 51E is a perspective view of the valve connector 5104 of FIGS. 51A-51D configured with its sealing gasket 5106 and its wiper ring 5108. FIG. 51F is an exploded view of the assembly of FIGS. 51A-51E showing port sizer 5102, valve connector 5104, sealing gasket 5106, wiper ring 5108, and actuation lever 5110.

The main difference between the assembly of FIGS. 51A-51E and the assembly of FIGS. 44A-49B is in the mechanism used to move the valve connector 5104 up and down within the port sizer 5102 to achieve the opened and closed configurations. Instead of the threading of FIGS. 44A-49B, the assembly of FIGS. 51A-51E has the actuation lever 5110 with an arm 5112 and a gear 5114 designed to engage with a corresponding gear rack 5116 of the valve connector 5104.

With the valve connector 5104 configured with the sealing gasket 5106 and the wiper ring 5108 (or, alternatively, and O-ring), the valve connector 5104 can be inserted into the port sizer 5102 all the way up to the closed configuration of FIG. 51C. At that point, the actuation lever 5110 can be inserted into the port sizer's side opening 5118 to engage the actuation lever's gear 5114 with the port sizer's gear rack 5116.

The actuation lever 5110 may be rotated (e.g., a quarter turn counterclockwise in the views of FIGS. 51A-51E) to partially retract the valve connector 5104 from the port sizer 5102 to achieve the opened configuration of FIG. 51D, which allows liquid to flow out of the bag assembly around the sealing gasket 5106 through the valve connector's holes 5120 and into the valve connector's passageway 5128.

After being opened by rotating the actuation lever 5110 to achieve the opened configuration shown in FIG. 51D, the assembly can be re-closed by de-rotating the actuation lever 5110 (e.g., a quarter turn clockwise) to re-achieve the closed configuration shown in FIG. 51C. In this way, the flow of liquid out of the bag assembly can once again be successfully regulated. Here, too, the valve connector 5104 and the sealing gasket 5106 close off the bag at the top of the canoe, again achieving zero dead leg and avoiding the dead-leg problem.

As shown in FIGS. 51B and 51F, the actuation lever's arm 5112 has a cable tie locking slot 5122 that enables the actuation lever 5110 to be secured using a cable tie (not shown) to the port sizer's closed-configuration cable tie locking slot 5124 to lock the assembly in the closed configuration of FIGS. 51A-51C. Analogously, the actuation lever's cable tie locking slot 5122 enables the actuation lever 5110 to be secured using a cable tie (not shown) to the port sizer's opened-configuration cable tie locking slot 5126 to lock the assembly in the opened configuration of FIG. 51D. Note that FIGS. 51A and 51C-E do not show the three locking slots 5122, 5124, and 5126 of FIGS. 51B and 51F.

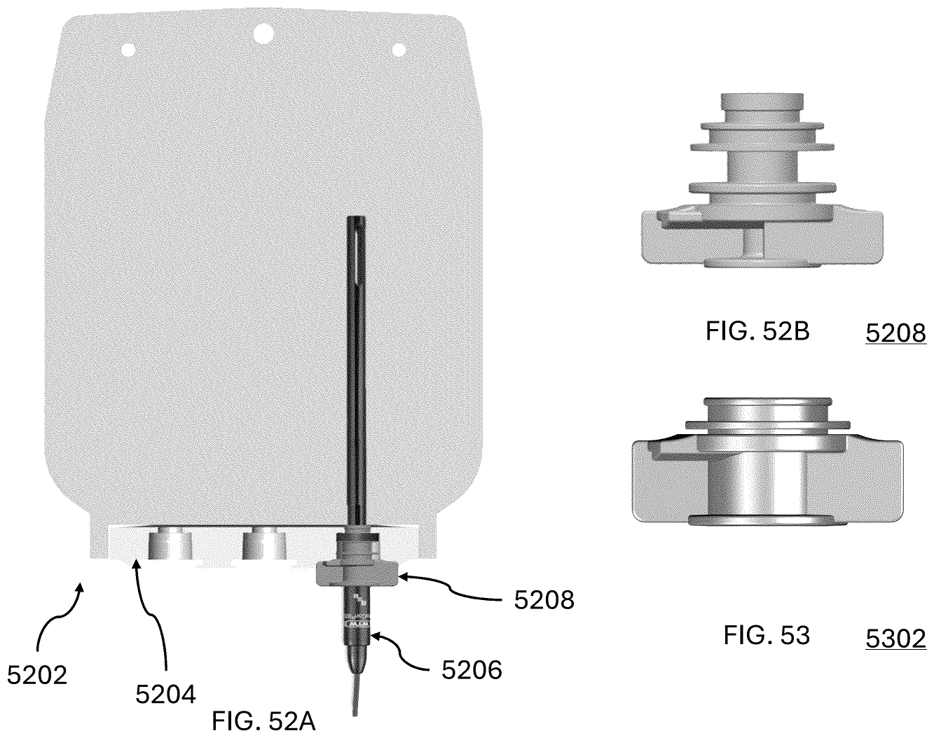

FIG. 52A is a cross-sectional view of a bag assembly 5202 having a three-port canoe 5204 configured with a probe 5206, for example, glued to a port fitting 5208 that engages with a port of the canoe 5204. FIG. 52B is a side view of the port fitting 5208 of FIG. 52A. FIG. 53 is a side view of an alternative port fitting 5302 that can be used to configure the probe 5206 to a different container, such as a 3D bag, bottle, or carboy. Instead of probes, other types of devices, such as suitable sensors, can be configured to suitable containers using the port fittings 5208 and 5302.

Although embodiments have been described in the context port fittings and port sizers that are mounted into ports of canoes for bag assemblies, such as canoe 3604 of bag assembly 3602 of FIG. 36, those skilled in the art will understand that those port fittings and port sizers may be mounted into the ports of other types of connectors for other types of containers, such as connectors similar to or identical to the connectors shown in FIGS. 26A-26E and 28.

Although the disclosure has been described in the context of port sizers (e.g., 108) that receive valve connectors (e.g., 110) having locking mechanisms (e.g., 606, 706, 708) that (i) prevent the valve connector from being removed from the port sizer after the valve connector has reached the closed position and (ii) prevent the valve connector from moving back to the closed position after the valve connector has reached the open position, the disclosure also covers embodiments in which (a) the valve connector can be moved back to the closed position from the open position and/or (b) the valve connect can be removed from the port sizer from the closed position. One possible implementation would involve corresponding threads on the outer diameter of the valve connector and the inner diameter of the port sizer that would allow selective clockwise or counterclockwise rotation of the valve connector with respect to the port sizer to move between closed and open positions. Such an implementation would involve modification and possibly removal of some of the existing features of the port sizer 108 and the valve connector 110, such as the locking mechanisms and keying features.

Although the disclosure has been described in the context of containers (e.g., 102) that receive interchangeable port sizers/fittings (e.g., 108/808), where the port fittings (e.g., 808) or valve connectors (e.g., 110) that are inserted into the port sizers (e.g., 108) have threaded tube barbs (e.g., 810) that receive threaded tube retainers (e.g., 1404), the disclosure also includes containers having permanent ports, where the permanent ports or analogous valve connectors that are inserted into the permanent ports have analogous threaded tube barbs that receive analogous threaded tube retainers.

Bag assemblies of the present disclosure may provide one or more of the following features:
Configurable with different port sizes as needed;
Fixed or flow controlling ports;
Standard fitment materials that are being used today;
Low extractable film-non protein binding;
Retainer system for tubing requiring no tools;
Modular construction;
With or without hanger option-snaps on;
One or more ports;
Can be port-size configured in minutes;
Flow lock or fixed ports;
Optional tube retention fittings-no tools required;
Canoe-shaped bag fitment with multiple (e.g., four) sealing bars for increased material welding/sealing surface area, which significantly reduces potential for leaks at weld points;
Concave shape on canoe, port sizer/fitting, and valve connector chamber-facing surfaces for efficient draining and no or low fluid retain;
Dust covers to reduce/eliminate possibility of contaminants entering chamber prior to final assembly (as well as storage and transport);
Filter-ready ports;
Modular designed ports can be configured with multiple sizes and multiple type industry connections and shapes on ends with no tools required to install;
Two-position valves having open and closed positions with flow stop ports-block flow until functioned;
Remains in lock closed position until deployed;
Retainer ready ports or standard ports;
Angle seal on port/retainer;
Modular hanger that can be added at any time;
Anti-slip features for ease of assembly with gloves;
Connector design provides for leak-proof tubing connections;
Variety of probe and sensors allowed to interface via the port sizer/fitting and canoe; and.
Fluid Sampling system with or without dosing chambers.

### Figure Labels

102 bag assembly
104 bag
106 bag hangar
108 port sizer
110 valve connector
112 bag chamber
114 three-port canoe
402 three-port canoe assembly
404 curved top surface of canoe 114
406 tethered safety clip
408 tether ring
410 tether clip
412 tether
414 dust cap
502 weld bar
504 canoe through-hole
506 canoe retention clip
508 canoe tapered end
602 port sizer O-ring groove
604 port sizer locking arm
606 port sizer locking clip
608 port sizer anti-slip grip
610 tether tab
612 port sizer stop.
614 port sizer channel
616 port sizer keyway
618 locking arm detent
620 port sizer rotation stop
622 locking clip connection
624 locking clip lower portion
626 locking clip upper portion
628 curved port sizer top surface
702A upper valve connector O-ring groove
702B lower valve connector O-ring groove
704 valve connector side port
706 valve connector “closed” locking tab
708 valve connector “opened” locking tab
710 valve connector stop
712 valve connector tube barb
714 valve connector channel
716 curved valve connector top surface
718 valve connector keyway guide
802 unthreaded, fixed port fitting
802A unthreaded, fixed port fitting
803 extended locking arm
804 fixed port fitting channel
806 port fitting tube barb
808 threaded, fixed port fitting
810 threaded tube barb
812 port fitting threading
814 fixed port fitting having a threaded “wye” tube connector
816 fixed port fitting having a threaded “tee” tube connector
818 retainer locking tab
902 luer-lock valve connector
904 luer lock
1002 block-out plug
1004 curved plug top surface
1006 plug O-ring groove
1008 plug locking arm
1102 “tee” valve connector
1104 “wye” valve connector
1106 “wye” valve connector
1108 valve connector housing
1202 female bag hangar component
1204 male bag hangar component
1206 bag hanger male element
1208 bag chamber opening
1210 bag hander female element
1302 bag probe
1400 three-port canoe assembly with tube retainer
1402 valve connector threading
1404 tube retainer
1802 tube
2002 straight edge of non-circular port sizer stop 612
2004 curved edge of non-circular port sizer stop 612
2006 straight portion of tether ring 408
2008 curved portion of tether ring 408
2202 threaded cylindrical portion of tube retainer 1404
2204 smooth frustum portion of the tube retainer 1404
2206 anti-rotation features of the tube retainer 1404
2208 tube retainer wing
2302 dust cap detent feature
2304 barbed end of threaded tube barb 810
2502 two-port canoe assembly
2504 two-port canoe
2602 single-fitting port
2604 fixed port fitting
2606 tube barb
2608 port welding flange
2610 retention clips
2612 locking arm
2614 septa gasket.
2616 two-fitting port
2618 four-fitting port
2702 septa gasket groove
2704 O-ring portion
2706 gasket portion
2802 cap
2902 dip tube
2904 dip tube fitting
2906 O-ring groove
2908 dip tube
2910 dip tube fitting
2912 septa gasket groove
3002 filling needle
3004 retainer-ready threaded connector
3006 filling needle mounting bar
3102 vented port fitting
3104 filter housing
3106 port fitting base
3202 canoe
3204 retention clips
3302 fixed tube-to-tube connector
3304 fixed tube-to-tube connector
3306 tube-to-tube connector with flow valve
3308 port sizer analog
3310 valve connector analog
3312 tube-to-tube connector analog
3314 probe
3402 fixed port fitting
3404 tube gripper
3502 reconfigurable valve connector/port sizer assembly
3504 valve connector
3506 port sizer
3508 valve connector threading
3602 bag assembly
3604 canoe
3606 bag
3608(1)-3608(3) port fittings
3610(1)-3610(3) dust caps
3612(1)-3612(3) ports
3614(1)-3614(3) tube barbs
3702 top of canoe 3604
3802 outer rim of a port 3612
3804 recessed, circular, inner seat of a port 3612
3806 raised, circular septa-sealing ring of a port 3612
3808 circular through-hole of a port 3612
3810 retention clip of a port 3612
3812 connecting structure for two adjacent retention clips 3810
3902 one-port 3D bag connector
3904 three-port 3D bag connector
3906 two-port bottle cap connector
4002 septa gasket
4004 septa gasket groove of the port fitting 3608(3)
4006 upper rim of the port fitting 3608(3)
4008 middle rim of the port fitting 3608(3)
4010 wings of the port fitting 3608(3)
4012 locking arm of the port fitting 3608(3)
4014 detent of the locking arm 4012
4016 lower rim of the port fitting 3608(3)
4018 top of the port fitting 3608(3)
4102 circular, O-ring portion of the septa gasket 4002
4104 circular, disk-shaped gasket portion of the septa gasket 4002
4106 circular through-hole of the septa gasket 4002
4108 circular recess of the septa gasket 4002
4202 cylindrical passage through the port fitting 3608(3)
4302 port fitting.
4304 threaded tube barb of the port fitting 4302
4306 external threading of the port fitting 4302
4308 tube retainer
4310 tube grippers of the port fitting 4302
4312 flexible tube
4402 port sizer
4404 valve connector
4406 sealing gasket
4408 wiper ring
4410 tube barb of the valve connector 4404
4412 internal threading of the port sizer 4402
4414 opening in the valve connector 4404
4416 passageway in the valve connector 4404
4504 septa gasket groove of the port sizer 4402.
4506 upper rim of the port sizer 4402
4508 middle rim of the port sizer 4402.
4510 wing of the port sizer 4402
4512 locking arm of the port sizer 4402
4516 lower rim of the port sizer 4402
4518 top of the port sizer 4402
4602 wing of the valve connector 4404
4604 external threading of the valve connector 4404
4606 top of the valve connector 4404
4608 wiper-ring groove of the valve connector 4404.
5002 port sizer
5004 valve connector
5006 locking tab of the valve connector 5004
5008 locking clips of the port sizer 5002
5010 wiper ring
5012 top of the valve connector 5004
5014 sealing gasket
5016 openings of the valve connector 5004
5018 passageway in the valve connector 5004
5102 port sizer
5104 valve connector
5106 sealing gasket
5108 wiper ring
5110 actuation lever
5112 arm of the actuation lever 5110
5114 gear of the actuation lever 5110
5116 gear rack of the valve connector 5104
5118 side opening of the port sizer 5102
5120 holes in the valve connector 5104
5122 cable tie locking slot of the actuation lever's arm 5112
5124 closed-configuration cable tie locking slot of the port sizer 5102
5126 opened-configuration cable tie locking slot of the port sizer 5102
5128 passageway of the valve connector 5104
5202 bag assembly
5204 three-port canoe
5206 probe
5208 port fitting
5302 port fitting

Those skilled in the art will understand that embodiments described in the context of port fittings and port sizers that connect to canoes can also be implemented in the context of port fittings and port sizers that connect to other types of ports, such as those for 3D bags, bottles, carboys, and caps.

In certain embodiments of the disclosure, an article of manufacture comprises a port fitting (e.g., 2604, 3608, 4302, 4402, 5002, 5102) for a container connector (e.g., 2602, 2616, 2618, 3604) having a connector port (e.g., 3612). The port fitting comprises (i) an upper rim (e.g., 4006) defining a septa gasket groove (e.g., 4004) and (ii) a pair of opposing locking arms (e.g., 4012). The septa gasket groove is configured to receive an O-ring portion (e.g., 4102) of a unitary septa gasket (e.g., 4002), while a gasket portion (e.g., 4104) of the unitary septa gasket rests on a top surface of the upper rim. With the port fitting fully mounted within the connector port, (i) the locking arms engage corresponding retention clips (e.g., 3810) of the connector port and (ii) the unitary septa gasket forms a leak-proof seal between the port fitting and the connector port.

In at least some of the above embodiments, the article further comprises the unitary septa gasket.

In at least some of the above embodiments, the article further comprises the container connector.

In at least some of the above embodiments, the connector port comprises an outer rim (e.g., 3802) and an inner seat (e.g., 3804). With the port fitting fully mounted within the connector port, (i) the O-ring portion of the unitary septa gasket abuts an inner surface of the outer rim and the port fitting and (ii) the gasket portion of the unitary septa gasket abuts the inner seat of the connector port and the top surface of the upper rim of the port fitting.

In at least some of the above embodiments, the inner seat of the connector port has a septa-sealing ring (e.g., 3806). With the port fitting fully mounted within the connector port, the septa-sealing ring forms a corresponding septa-seal depression in the gasket portion (e.g., 4104) of the septa gasket (e.g., 4002).

In at least some of the above embodiments, the port fitting is a fixed port fitting having a tube barb (e.g., 3614, 4304).

In at least some of the above embodiments, the tube barb is a threaded tube barb (e.g., 4304) configured to receive a threaded tube retainer (e.g., 4308).

In at least some of the above embodiments, the port fitting is configured to receive a valve connector (e.g., 4404, 5004, 5104) at (i) a closed position corresponding to a closed configuration that prevents liquid from flowing through the connector port, the port fitting, and the valve connector and (ii) an opened position corresponding to an opened configuration that enables liquid to flow through the connector port, the port fitting, and the valve connector.

In at least some of the above embodiments, in the closed configuration, the port fitting and the valve connector achieve zero dead leg.

In at least some of the above embodiments, the port fitting (e.g., 4402) has internal threading (e.g., 4412) and the valve connector (e.g., 4404) has external threading (e.g., 4604) configured to engage the internal threading of the port fitting to move the valve connector relative to the port fitting between the closed position and the opened position.

In at least some of the above embodiments, the port fitting (e.g., 5002) has opposing locking clips (e.g., 5008) and the valve connector (e.g., 5004) has at least one locking tab (e.g., 5006) configured to engage the locking clips of the port fitting at the at least one locking tab at the closed position.

In at least some of the above embodiments, the port fitting (e.g., 5102) has a side opening (e.g., 5118) configured to receive a gear (e.g., 5114) of an actuation lever (e.g., 5110) and the valve connector (e.g., 5104) has a gear track (e.g., 5116) configured to engage the gear of the actuation lever to move the valve connector relative to the port fitting between the closed position and the opened position.

In at least some of the above embodiments, the article further comprises the actuation lever.

While this disclosure includes references to illustrative embodiments, this specification is not intended to be construed in a limiting sense. Various modifications of the described embodiments, as well as other embodiments within the scope of the disclosure, which are apparent to persons skilled in the art to which the disclosure pertains are deemed to lie within the principle and scope of the disclosure, e.g., as expressed in the following claims.

Unless explicitly stated otherwise, each numerical value and range should be interpreted as being approximate as if the word “about” or “approximately” preceded the value or range.

It will be further understood that various changes in the details, materials, and arrangements of the parts which have been described and illustrated in order to explain the nature of this disclosure may be made by those skilled in the art without departing from the scope of the disclosure, e.g., as expressed in the following claims.

Reference herein to “one embodiment” or “an embodiment” means that a particular feature, structure, or characteristic described in connection with the embodiment can be included in at least one embodiment of the disclosure. The appearances of the phrase “in one embodiment” in various places in the specification are not necessarily all referring to the same embodiment, nor are separate or alternative embodiments necessarily mutually exclusive of other embodiments. The same applies to the term “implementation.”

Unless otherwise specified herein, the use of the ordinal adjectives “first,” “second,” “third,” etc., to refer to an object of a plurality of like objects merely indicates that different instances of such like objects are being referred to, and is not intended to imply that the like objects so referred-to have to be in a corresponding order or sequence, either temporally, spatially, in ranking, or in any other manner.

Also for purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. Conversely, the terms “directly coupled,” “directly connected,” etc., imply the absence of such additional elements. The same type of distinction applies to the use of terms “attached” and “directly attached,” as applied to a description of a physical structure. For example, a relatively thin layer of adhesive or other suitable binder can be used to implement such “direct attachment” of the two corresponding components in such physical structure.

The described embodiments are to be considered in all respects as only illustrative and not restrictive. In particular, the scope of the disclosure is indicated by the appended claims rather than by the description and figures herein. All changes that come within the meaning and range of equivalency of the claims are to be embraced within their scope.

In this specification including any claims, the term “each” may be used to refer to one or more specified characteristics of a plurality of previously recited elements or steps. When used with the open-ended term “comprising,” the recitation of the term “each” does not exclude additional, unrecited elements or steps. Thus, it will be understood that an apparatus may have additional, unrecited elements and a method may have additional, unrecited steps, where the additional, unrecited elements or steps do not have the one or more specified characteristics.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

All documents mentioned herein are hereby incorporated by reference in their entirety or alternatively to provide the disclosure for which they were specifically relied upon.

The embodiments covered by the claims in this application are limited to embodiments that (1) are enabled by this specification and (2) correspond to statutory subject matter. Non-enabled embodiments and embodiments that correspond to non-statutory subject matter are explicitly disclaimed even if they fall within the scope of the claims.

While preferred embodiments of the disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the technology of the disclosure. It is intended that the following claims define the scope of the invention and that methods and structures within the scope of these claims and their equivalents be covered thereby.

What is claimed is:

1. An article of manufacture comprising a port fitting (e.g., 2604, 3608, 4302, 4402, 5002, 5102) for a container connector (e.g., 2602, 2616, 2618, 3604) having a connector port (e.g., 3612), the port fitting comprising:
an upper rim (e.g., 4006) defining a septa gasket groove (e.g., 4004); and
a pair of opposing locking arms (e.g., 4012), wherein:
the septa gasket groove is configured to receive an O-ring portion (e.g., 4102) of a unitary septa gasket (e.g., 4002), while a gasket portion (e.g., 4104) of the unitary septa gasket rests on a top surface of the upper rim; and
with the port fitting fully mounted within the connector port, (i) the locking arms engage corresponding retention clips (e.g., 3810) of the connector port and (ii) the unitary septa gasket forms a leak-proof seal between the port fitting and the connector port.

2. The article of claim 1, further comprising the unitary septa gasket.

3. The article of claim 1, further comprising the container connector.

4. The article of claim 3, wherein:
the connector port comprises an outer rim (e.g., 3802) and an inner seat (e.g., 3804); and
with the port fitting fully mounted within the connector port, (i) the O-ring portion of the unitary septa gasket abuts an inner surface of the outer rim and the port fitting and (ii) the gasket portion of the unitary septa gasket abuts the inner seat of the connector port and the top surface of the upper rim of the port fitting.

5. The article of claim 4, wherein:
the inner seat of the connector port has a septa-sealing ring (e.g., 3806); and
with the port fitting fully mounted within the connector port, the septa-sealing ring forms a corresponding septa-seal depression in the gasket portion (e.g., 4104) of the septa gasket (e.g., 4002).

6. The article of claim 1, wherein the port fitting is a fixed port fitting having a tube barb (e.g., 3614, 4304).

7. The article of claim 6, wherein the tube barb is a threaded tube barb (e.g., 4304) configured to receive a threaded tube retainer (e.g., 4308).

8. The article of claim 1, wherein the port fitting is configured to receive a valve connector (e.g., 4404, 5004, 5104) at (i) a closed position corresponding to a closed configuration that prevents liquid from flowing through the connector port, the port fitting, and the valve connector and (ii) an opened position corresponding to an opened configuration that enables liquid to flow through the connector port, the port fitting, and the valve connector.

9. The article of claim 8, wherein, in the closed configuration, the port fitting and the valve connector achieve zero dead leg.

10. The article of claim 8, further comprising the valve connector.

11. The article of claim 10, wherein:
the port fitting (e.g., 4402) has internal threading (e.g., 4412); and
the valve connector (e.g., 4404) has external threading (e.g., 4604) configured to engage the internal threading of the port fitting to move the valve connector relative to the port fitting between the closed position and the opened position.

12. The article of claim 10, wherein:
the port fitting (e.g., 5002) has opposing locking clips (e.g., 5008); and
the valve connector (e.g., 5004) has at least one locking tab (e.g., 5006) configured to engage the locking clips of the port fitting at the at least one locking tab at the closed position.

13. The article of claim 10, wherein:
the port fitting (e.g., 5102) has a side opening (e.g., 5118) configured to receive a gear (e.g., 5114) of an actuation lever (e.g., 5110); and
the valve connector (e.g., 5104) has a gear track (e.g., 5116) configured to engage the gear of the actuation lever to move the valve connector relative to the port fitting between the closed position and the opened position.

14. The article of claim 13, further comprising the actuation lever.
